(19) United States
(12) Patent Application Publication
(10) Pub. No.: US 2014/0134170 A1
(43) Pub. Date: May 15, 2014

(54) USE OF INHIBITORS OF EGFR-FAMILY RECEPTORS IN THE TREATMENT OF HORMONE REFRACTORY BREAST CANCERS

(71) Applicants: Gabriela Garcia, Roslindale, MA (US); William Kubasek, Belmont, MA (US); Gavin Macbeath, Danvers, MA (US); Victor Moyo, Concord, MA (US)

(72) Inventors: Gabriela Garcia, Roslindale, MA (US); William Kubasek, Belmont, MA (US); Gavin Macbeath, Danvers, MA (US); Victor Moyo, Concord, MA (US)

(73) Assignee: MERRIMACK PHARMACEUTICALS, INC., Cambridge, MA (US)

(21) Appl. No.: 14/004,598

(22) PCT Filed: Mar. 12, 2012
(86) PCT No.: PCT/US12/28792

§ 371 Date: Nov. 20, 2013

Related U.S. Application Data
(60) Provisional application No. 61/451,848, filed on Mar. 11, 2011.
(60) Provisional application No. 61/604,281, filed on Feb. 28, 2012.

(51) Int. Cl. A61K 39/395; C07K 16/32; A61K 31/5685; C07K 16/46; A61K 45/06; A61K 31/4196

(52) U.S. Cl. CPC A61K 39/39558; A61K 45/06; A61K 31/4196; A61K 31/5685; C07K 16/468; C07K 16/32
USPC 424/136.1; 424/138.1; 530/387.3; 530/387.7; 435/375

(57) ABSTRACT

Provided are methods of suppressing growth of hormone refractory breast tumors by contacting tumor cells with an ErbB3 inhibitor, preferably an anti-ErbB3 antibody. Also provided are methods for treating hormone refractory breast cancer in a patient by administering to the patient an inhibitor of heregulin binding to ErbB3 or to ErbB2/ErbB3 heterodimer, which inhibitor is an anti-ErbB3 antibody or an anti-ErbB2 antibody. The treatment methods can further comprise selecting a patient having a hormone refractory breast cancer and then administering the inhibitor to the patient. The treatment methods may also comprise administering an estrogen receptor antagonist, or an aromatase inhibitor to the patent and may at further comprise administering to the patient at least one additional anti-cancer agent that is not an ErbB3 inhibitor, an estrogen receptor antagonist, or an aromatase inhibitor to the patient in combination with the ErbB3 inhibitor.

### BACKGROUND

In women, breast cancer is among the most common cancers and is the fifth most common cause of cancer deaths. Due to the heterogeneity of breast cancers, 10-year progression free survival can vary widely with stage and type, from 98% to 10%. Different forms of breast cancers can have remarkably different biological characteristics and clinical behavior. Thus, classification of a patient's breast cancer has become a critical component for determining a treatment regimen. For example, along with classification of histological type and grade, breast cancers now are routinely evaluated for expression of hormone receptors (estrogen receptor (ER) and progesterone receptor (PR) and for expression of HER2 (ErbB2), since a number of treatment modalities are currently available that target hormone receptors or HER2. Other cancers, e.g., uterine or ovarian cancers, may be similarly characterized. ER and PR are both nuclear receptors (i.e., they are predominantly located at cell nuclei, rather than the cell surface) and small molecule inhibitors that directly or indirectly target ER and/or PR have been developed. HER2, or human epidermal growth factor receptor type 2, is a receptor normally located on the cell surface and antibodies that target HER2 have been developed as therapeutics. HER2 is the only member of the EGFR family (which also includes HER1 (EGFR), HER3 (ErbB3) and HER4 (ErbB4)) that is not capable of binding to an activating ligand on its own. Thus HER2 is only functional as a receptor when incorporated into a heterodimeric receptor complex with another EGFR family member, such as HER3. Cancers classified as expressing the estrogen receptor (estrogen receptor positive, “ER+”) may be treated with an ER antagonist such as tamoxifen. Similarly, cancers classified as expressing high levels of the HER2 may be treated with an anti-HER2 antibody, such as trastuzumab, or with a HER2-active receptor tyrosine kinase inhibitor such as lapatinib (which also inhibits EGFR tyrosine kinase) or AG879.

Tamoxifen has been used as therapy against ER+ breast cancer for decades and now represent a standard component of front-line therapy for ER+ breast cancers. Tamoxifen is a member of the class of selective estrogen receptor modulators (e.g., raloxifene, toremifene and fulvestrant), of which tamoxifen, toremifene and fulvestrant are estrogen receptor antagonists and raloxifene has agonist activity in bone and antagonist activity in breast and uterine cancers. These antagonist drugs specifically block the hormonal activation of the estrogen receptor and are effective therapeutic agents for the treatment of ER+ breast cancers that have not become hormone refractory. Tamoxifen, for example, induces remissions in over half of ER+ breast cancer patients upon initial treatment. The long term utility of hormone receptor blockade is limited by the phenomenon of the development of hormone refractory tumor characteristics following extended treatment. Most treated tumors eventually become hormone refractory in that they become tamoxifen resistant.

Thus, hormonal blockade with hormone antagonists and other hormone modulatory drugs such as aromatase inhibitors (e.g., exemestane, anastrozole, letrozole, anastrozole, vorozole, formestane and fadrozole), which block estrogen synthesis, can delay progression of ER+ tumors, but the frequent development of resistance to such hormone modulatory drugs has created a longstanding need for anti-cancer therapeutic agents that are effective against hormone refractory ER+ cancers. The present disclosure addresses this need and provides additional benefits.

### SUMMARY

Provided herein are methods for treating hormone refractory breast cancers (e.g., tumors), including estrogen receptor positive and estrogen receptor negative hormone refractory breast cancers, as well as pharmaceutical compositions that can be used in such methods. The methods and compositions are based, at least in part, on the discovery that ErbB3 inhibition can suppress the growth of hormone refractory breast cancer cells. In particular, administration of anti-ErbB3 antibody is believed to suppress the growth of hormone refractory breast cancer cells. Furthermore, it has now been discovered that heregulin activation of ErbB2/ErbB3 heterodimers can in turn activate (by causing the phosphorylation of) estrogen receptors, a phenomenon that is believed to play a role in the development of resistance to hormone modulatory drugs in ER+ tumors. Thus, also provided herein are methods and compositions for inhibiting the activation of estrogen receptors by inhibiting the binding of heregulin to ErbB2/ErbB3 heterodimers. Such methods may be beneficially practiced in combination with co-administration of one or more estrogen receptor modulatory drugs as described herein.

Accordingly, use of an ErbB3 inhibitor (e.g., use thereof for the manufacture of a medicament) for the treatment of hormone refractory breast cancer is provided. In another aspect, a method is disclosed of suppressing growth of a hormone refractory breast cancer tumor (optionally an estrogen receptor positive hormone refractory breast cancer tumor), the method comprising contacting the tumor with an effective amount of an ErbB3 inhibitor. In another aspect, a method of suppressing growth of a hormone refractory breast cancer tumor (optionally an estrogen receptor positive hormone refractory breast cancer tumor) in a patient is provided, the method comprising administering to the patient an effective amount of an ErbB3 inhibitor. In yet another aspect, a method of treating a patient for a hormone refractory breast cancer tumor (optionally an estrogen receptor positive hormone refractory breast cancer tumor) is provided, the method comprising administering to the patient an effective amount of an ErbB3 inhibitor. In still another aspect, a method of treating a breast cancer tumor in a patient is provided, the method comprising: selecting a patient with a hormone refractory breast cancer tumor (optionally an estrogen receptor positive hormone refractory breast cancer tumor); and administering to the patient an effective amount of an ErbB3 inhibitor.

In an exemplary embodiment, the ErbB3 inhibitor is an anti-ErbB3 antibody. An exemplary anti-ErbB3 antibody is Ab #6, comprising V_{H} and/or V_{L} regions comprising the amino acid sequences set forth in SEQ ID NOs: 1 and 2, respectively. Another exemplary anti-ErbB3 antibody is an antibody comprising, optionally in amino terminal to carboxy terminal order, V_{H} CDR1, 2 and 3 sequences as shown in SEQ ID NOs: 3-5, respectively, and, optionally in amino terminal to carboxy terminal order, V_{L} CDR1, 2 and 3 sequences as shown in SEQ ID NOs: 6-8, respectively. In another embodiment, the anti-ErbB3 antibody has heavy and light chains comprising the amino acid sequences set forth in SEQ ID NOs 42 and 43, respectively. In other embodiments, the anti-ErbB3 antibody is Ab #3 (comprising V_{H} and V_{L} sequences as shown in SEQ ID NOs: 9 and 10, respectively), Ab #14 (comprising V_{H} and V_{L} sequences as shown in SEQ ID NOs: 17 and 18, respectively), Ab #17 (comprising V_{H} and V_{L} sequences as shown in SEQ ID NOs: 25 and 26, respectively) or Ab #19 (comprising V_{H} and V_{L} sequences as shown in SEQ ID NOs: 33 and 34, respectively). In another embodiment, administration of the anti-ErbB3 antibody inhibits growth or invasiveness or metastasis of the tumor.

In another aspect, the treatment methods provided herein further comprise co-administering to the patient at least one additional anti-cancer agent that is not an ErbB3 inhibitor. In one embodiment, the at least one additional anti-cancer agent comprises at least one chemotherapeutic drug, such as a drug(s) selected from the group consisting of platinum-based chemotherapy drugs, taxanes, tyrosine kinase inhibitors, serine/threonine protein kinase inhibitors, anti-EGFR antibodies, anti-ErbB2 antibodies, bispecific anti-ErbB2/ErbB3 antibodies, and combinations thereof.

In another embodiment, the at least one additional anti-cancer agent comprises an EGFR inhibitor, such as an anti-EGFR antibody or a small molecule inhibitor of EGFR signaling. A preferred anti-EGFR antibody comprises cetuximab. Other examples of anti-EGFR antibodies include MM-151, Sym004, matuzumab, panitumumab, nimotuzumab and mAb 806. An exemplary small molecule inhibitor of EGFR signaling comprises gefitinib. Other examples of useful small molecule inhibitors of EGFR signaling include but are not limited to afatinib, lapatinib, canertinib, erlotinib HCL, pelitinib, PKI-166, PD-158780, and AG 1478.

In yet another embodiment, the at least one additional anti-cancer agent comprises a vascular endothelial growth factor (VEGF) inhibitor. An exemplary VEGF inhibitor comprises an anti-VEGF antibody, such as the bevacizumab antibody. In still another embodiment, the at least one additional anti-cancer agent comprises either or both of an estrogen receptor antagonist and an aromatase inhibitor. Examples of estrogen receptor antagonists include raloxifene, tamoxifen, afimoxifene (4-hydroxytamoxifen), arzoxifene, lasofoxone, toremifene and fulvestrant. Examples of aromatase inhibitors include but are not limited to exemestane, anastrozole, letrozole, aminoglutethimide, testolactone, vorozole, formestane and fadrozole. In one embodiment, the aromatase inhibitor is letrozole. In still another embodiment, the at least one additional anti-cancer agent comprises a serine/threonine protein kinase inhibitor, such as a mammalian target of rapamycin (mTOR) inhibitor, a phosphatidylinositol-3-kinase (PI3K) inhibitor, or a mitogen activated kinase kinase (MEK) inhibitor. Examples of mTOR inhibitors include but are not limited to temsirolimus, everolimus, sirolimus, or ridaforolimus. Examples of PI3K inhibitors include but are not limited to CAL101 and PX-866, both of which are currently being tested in clinical trials. Examples of MEK inhibitors include but are not limited to XL518, CI-1040, PD035901, selumetinib, and GSK1120212. In one embodiment, the at least one additional anti-cancer agent comprises either or both of an mTOR inhibitor and an aromatase inhibitor. In one embodiment, the at least one anti-cancer agent comprises everolimus and exemestane. In yet another embodiment, the at least one additional anti-cancer agent comprises an IGF1R inhibitor. Examples of IGF1R inhibitors include dalotuzumab, AMG-479, R1507, figitumumab, IMC-A12, XL228, BMS-754807 and MM-141.

In one embodiment, the hormone refractory breast cancer is ER+.

In a further aspect, provided herein are methods for inhibiting heregulin-mediated activation of estrogen receptors in tumor cells, said method comprising 1) selecting a human patient who has been treated for a malignancy with an anti-estrogen therapy and has become resistant to such therapy, which patient has a malignant tumor, which tumor, by analysis of a tumor biopsy taken from the patent after the patient has become resistant, is estrogen receptor positive and overexpresses HER2, and which activation comprises phosphorylation of estrogen receptors, and 2) administering to the patient so selected an antibody that inhibits heregulin binding to ErbB2/ErbB3 heterodimer, wherein the antibody is administered at a dosage that yields a concentration of the antibody in the patient's bloodstream that is a sufficient concentration to inhibit heregulin-induced estrogen receptor phosphorylation in MCF7 cells in vitro by at least 20%, at least 30%, at least 40%, at least 50%, at least 60%, or at least 70%, wherein said administration at said dosage is effective to treat the tumor.

The cell may be in a tumor that, by biopsy, is ER⁺ and HER2⁺⁺ or HER2⁺⁺⁺, or that contains at least 0.02 pg HRG/μg of protein (e.g., by ELISA), or is HER2 FISH-positive. The inhibition is accomplished by introducing into the extracellular fluid an antibody that inhibits heregulin binding to ErbB2/ErbB3 heterodimer. In one embodiment the tumor is a malignant tumor.

Non-limiting examples of types of tumors to be treated include cancers of the breast, ovary, lung, or skin (e.g., melanoma).

The tumor may be in a patient and the antibody introduced into the bloodstream by administration to the patient of an amount of the antibody that is effective to yield the sufficient concentration of the antibody in the bloodstream. The administration may be by intravenous injection or infusion. In one embodiment the antibody may be an anti-HER3 (anti-ErbB3) antibody, e.g., an antibody having V_{H} and/or V_{L} regions comprising the amino acid sequences set forth in SEQ ID NOs: 1 and 2, respectively. In another embodiment the antibody may be an anti-ErbB3 antibody comprising V_{H} and/or V_{L} regions comprising the amino acid sequences set forth in SEQ ID NOs: 42 and 43, respectively. The antibody may be an anti-HER2 (anti-ErbB2) antibody, e.g., C6.5, C6.5 diabody, or pertuzumab. The antibody may also be an anti-ErbB2/anti-ErbB3 bispecific antibody. A number of bispecific anti-ErbB2/anti-ErbB3 antibodies that are scFv human serum albumin (HSA) conjugates are described in US patent publication 20110059076, and PCT publication number WO2009/126920, each of which discloses B2B3-1 and other bispecific anti-ErbB2/antiErbB3 antibodies that are scFv HSA conjugates and that are suitable for use in the methods and compositions provided herein, including ALM, A5-HSA-ML3.9, A5-HSA-B1D2, B12-HSA-B1D2, A5-HSA-F5B6H2, H3-HSA-F5B6H2, F4-HSA-F5B6H2, and H3-HSA-B1D2. In one embodiment, the bispecific antibody comprises SEQ ID NO:44. Other suitable bispecific anti-ErbB2/antiErbB3 antibodies are disclosed and claimed in U.S. Pat. Nos. 7,332,580 and 7,332,585. Preferably, administration of the antibody inhibits growth or invasiveness or metastasis of the tumor.

Accordingly, an ErbB3 inhibitor (e.g., an anti-ErbB3 antibody) or an anti-ErbB2 antibody or a bispecific anti-ErbB2/ErbB3 antibody is provided (e.g., use thereof for the manufacture of a medicament) for the inhibition of heregulin mediated estrogen receptor activation, and also or alternately for the treatment of hormone refractory breast cancer (or another hormone refractory cancer such as ovarian cancer, uterine cancer, or cervical cancer) or of aromatase resistant estrogen receptor positive cancer such as breast cancer, ovarian cancer, uterine cancer, or cervical cancer, is disclosed. In an additional embodiment, an ErbB3 inhibitor, e.g., an anti-ErbB3 antibody or an anti-ErbB2 antibody or a bispecific anti-ErbB2/ErbB3 antibody is provided (e.g., use thereof for the manufacture of a medicament) for use in the treatment of an estrogen receptor positive cancer (e.g., breast cancer, ovarian cancer, uterine cancer, or cervical cancer) in combination therapy with an aromatase inhibitor. Such combinations retard or prevent the development of hormone resistance in cancers treated with such combinations. In additional embodiments the method further comprises co-administration of either or both of an estrogen receptor antagonist and an aromatase inhibitor. In further aspects herein provided are compositions for inhibition of heregulin-mediated activation of estrogen receptor, said inhibition following selection of a human patient who has been treated for malignancy with an anti-estrogen therapy and has become resistant to such therapy, which patient has a malignant tumor, which tumor, by analysis of a tumor biopsy taken from the patent after the patient has become resistant, is estrogen receptor positive and overexpresses ErbB2, and which activation comprises phosphorylation of estrogen receptors, said composition comprising: an anti-ErbB3 antibody that inhibits heregulin binding to ErbB2/ErbB3 heterodimer; an anti-ErbB2 antibody that binds to ErbB2 and inhibits heregulin binding to ErbB2/ErbB3 heterodimer (e.g., pertuzumab); or comprising an anti-ErbB2/antiErbB3 bispecific antibody that inhibits heregulin binding to ErbB2/ErbB3 heterodimer (e.g., the antibody comprising SEQ ID NO:44 (also referred to as SEQ ID NO:16 in U.S. patent publication No. 20110059076). In some embodiments the cancer is a hormone refractory estrogen-receptor positive cancer.

In one embodiment, each of these compositions optionally comprises one or more of an estrogen receptor antagonist and an aromatase inhibitor. Examples of estrogen receptor entagonists include raloxifene, tamoxifen, afimoxifene (4-hydroxytamoxifen), arzoxifene, lasofoxone, toremifene and fulvestrant. Examples of aromatase inhibitors include exemestane, anastrozole, letrozole, aminoglutethimide, testolactone, vorozole, formestane and fadrozole. In an exemplary embodiment, the aromatase inhibitor is letrozole.

In another embodiment, each of these compositions optionally comprises one or more of an mTOR inhibitor and an aromatase inhibitor. Examples of mTOR inhibitors include temsirolimus, everolimus, sirolimus, or ridaforolimus. In an exemplary embodiment, the mTOR inhibitor is everolimus. Examples of aromatase inhibitors include exemestane, anastrozole, letrozole, aminoglutethimide, testolactone, vorozole, formestane and fadrozole. In an exemplary embodiment, the aromatase inhibitor is exemestane.

In another embodiment, each of these compositions optionally comprises one or more of a MEK inhibitor, a PI3K inhibitor, and an IGF-1R inhibitor.

### BRIEF DESCRIPTION OF THE DRAWINGS

FIG. 1 comprises images of western blots of a gel of lysates from untreated control cells (“C”), cells pretreated with (“MM121”) or without pretreatment with MM-121 that were stimulated with heregulin beta 1 (“HRG”), betacellulin (“BTC”), or estrogen (“E2”). The top panel shows results from a blot probed with an antibody specific to phosphorylated ErbB3 (pErbB3), the middle panel shows a blot probed with an antibody specific to phosphorylated (ser 167 and ser 118) estrogen receptor alpha (pER), and the bottom panel shows a blot probed with an antibody specific to glyceraldehyde-3-phosphate dehydrogenase (GAPDH) as a loading control.

FIG. 2 is a graph showing densitometry results from the relevant (pER) band in each of the control (“Con.”), heregulin (“HRG”), betacellulin (“BTC”), heregulin plus MM-121 (“HRG+MM121”) and betacellulin plus MM-121 (“BTC+MM121”) lanes in FIG. 1. Band density was normalized to GAPDH density and the normalized density (Y-axis) is shown for control and stimulated cells (both with and without MM-121 pretreatment) as indicated on the X-axis. The arrowhead between the HRG and BTC lanes indicates the pER band in the HRG lane.

FIG. 3 is a graph showing tumor volume (y-axis) over time (in weeks, x axis) in a letrozole resistant mouse xenograft model. Data are shown for mice treated with: PBS as a control (square), MM-121 alone (“MM”, triangle), letrozole alone (“Let”, upside down triangle), and the combination of MM-121 and letrozole (MM+Let, diamond). At the 14 week mark, the letrozole mice were split into three groups: letrozole alone (“Let”, upside down triangle), MM-121 alone (“Let→MM”, circle) and the combination of MM-121 and letrozole (“Let→MM+Let”, star).

### DETAILED DESCRIPTION

Provided herein are methods for treating hormone refractory breast cancers and other ER+ cancers, particularly those that overexpress HER2. Also provided are pharmaceutical compositions for, and uses thereof in, such treatment. As described further in the Examples, it is believed that ErbB3 inhibitors, e.g., anti-ErbB3 antibodies, or other antibodies that can inhibit the binding of heregulin to ErbB2/ErbB3 heterodimers, are able to suppress one or more of the growth, invasiveness and metastasis of hormone refractory breast cancer cells in vivo. Accordingly, provided are methods and compositions and uses thereof for suppressing the growth invasiveness or metastasis of hormone refractory breast cancers (e.g., estrogen receptor positive hormone refractory breast cancers), as well as methods and compositions for treating such breast cancers in patients, e.g., with an ErbB3 inhibitor.

ER+ cancers exemplify candidates for therapy regimens that include anti-estrogen agents. Such cancers may include but are not limited to certain breast, ovarian, uterine, endometrial, lung, bone, brain, bladder, liver and urogenital cancers.

A cancer may be an ErbB2 gene amplified cancer and/or an ErbB2 expressing (HER2+) or overexpressing (HER2++, HER2+++) cancer. ErbB2, also known as HER2 or Neu, is a cell surface transmembrane receptor protein that generates intracellular signals (e.g., upon ligand activation) via its intracellular tyrosine kinase activity. In excess, such signals can promote oncogenesis e.g., by triggering cell division. The ErbB2 gene is amplified and/or overexpressed in many types of human malignancies, including but not limited to breast, ovarian, endometrial, pancreatic, colorectal, prostate, salivary gland, skin, kidney, and lung. ErbB2 overexpressing cancers are designated a HER2+++ or HER2++ depending on the level of ErbB2 overexpression, with HER2+++ indicating the highest levels of HER2 expression. HER2+++ and HER2++ status are typically determined by an immunoassay such as immunohistochemistry (IHC), e.g., Herceptest®. According to guidelines provided by the College of American Pathologists (CAP) and the American Society of Clinical Oncology (ASCO), a tumor designated HER2 negative is a tumor in which an IHC test shows no staining or membrane staining in <30% of tumor cells; a tumor is designated “HER2”+ if an IHC test results in faint membrane staining in >30% of tumor cells, wherein only part of membrane is stained; a tumor is designated “HER2++” if an IHC assay results in weak or moderate (complete) membrane staining in >30% of tumor cells; and a tumor is designated “HER2+++” if an IHC test results in a uniform, intense stain of >30% of the tumor cells. ErbB2 gene amplification is may be determined by, e.g., FISH (fluorescence in situ hybridization), with HER2-amplified cancer cells being those that have more than two HER2 gene copies being HER2-amplified, and cells and/or tumors comprising HER2-amplified cancer cells being referred to as “FISH positive.”

### DEFINITIONS

As used herein, the term “hormone refractory breast cancer” refers to breast cancer that is resistant to the effects of anti-hormone therapy. A hormone refractory breast cancer is an estrogen receptor positive breast cancer that is either de novo resistant to endocrine therapy or acquires resistance while on treatment. About 25-50% of hormone-receptor-positive breast cancers are de novo resistant to endocrine therapy, and essentially all metastatic breast cancers develop acquired resistance.

As used herein, the term “estrogen receptor positive” (ER+) refers to tumors (e.g., carcinomas), typically breast tumors, in which the tumor cells score positive (i.e., using conventional histopathology methods) for estrogen receptor (ER). According to recommendations provided by CAP and ASCO, a tumor is ER+ if at least 1% of the tumor cells tested (e.g., by immunohistochemistry) score ER positive.

The terms “ErbB3” and “HER3,” as used interchangeably herein, refer to human ErbB3 protein, as described in U.S. Pat. No. 5,480,968.

The terms “ErbB2,” “HER2,” and “HER2 receptor,” as used interchangeably herein, refer to the protein product of the human neu oncogene, also referred to as the ErbB2 oncogene or the HER2 oncogene.

As used herein, the term “ErbB3 inhibitor” is intended to include therapeutic agents that inhibit, downmodulate, suppress or downregulate activity of ErbB3. The term is intended to include chemical compounds, such as small molecule inhibitors, and biologic agents, such as antibodies, interfering RNA (shRNA, siRNA), soluble receptors and the like. An exemplary ErbB3 inhibitor is an anti-ErbB3 antibody.

An “antibody,” as used herein is a protein consisting of one or more polypeptides comprising binding domains substantially encoded by immunoglobulin genes or fragments of immunoglobulin genes, wherein the protein immunospecifically binds to an antigen. The recognized immunoglobulin genes include the kappa, lambda, alpha, gamma, delta, epsilon and mu constant region genes, as well as myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. A typical immunoglobulin structural unit comprises a tetramer that is composed of two identical pairs of polypeptide chains, each pair having one “light” (about 25 kD) and one “heavy” chain (about 50-70 kD). “V_{L}” and V_{H}” refer to the variable regions of these light and heavy chains respectively.

Antibodies include intact immunoglobulins as well as antigen-binding fragments thereof, which may be produced by digestion with various peptidases, or synthesized de novo either chemically or using recombinant DNA expression technology. Such fragments include, for example, F(ab)₂ dimers and Fab monomers. Useful antibodies include single chain antibodies (antibodies that exist as a single polypeptide chain), e.g., single chain Fv antibodies (scFv) in which a V_{H} and a V_{L} chain are joined together (directly or through a peptide linker) to form a continuous polypeptide.

“Immunospecific” or “immunospecifically” refer to antibodies that bind via domains substantially encoded by immunoglobulin genes or fragments of immunoglobulin genes to one or more epitopes of a protein of interest, but which do not substantially recognize and bind other molecules in a sample containing a mixed population of antigenic molecules. Typically, an antibody binds immunospecifically to a cognate antigen with a K_{d} with a value of no greater than 50 nM, as measured by a surface plasmon resonance assay or a cell binding assay. The use of such assays is well known in the art, and is described in Example 3, below.

An “anti-ErbB3 antibody” is an antibody that immunospecifically binds to the ectodomain of ErbB3 and an “anti-ErbB2 antibody” is an antibody that immunospecifically binds to the ectodomain of ErbB2. The antibody may be an isolated antibody. Such binding to ErbB3 or ErB2 exhibits a K_{d} with a value of no greater than 50 nM as measured by a surface plasmon resonance assay or a cell binding assay. Exemplary anti-ErbB3 antibodies inhibit EGF-like ligand mediated phosphorylation of ErbB3, e.g., anti-ErbB2 antibodies that inhibit the binding of heregulin to ErbB2/ErbB3 heterodimers. EGF-like ligands include EGF, TGFα, betacellulin, heparin-binding epidermal growth factor, biregulin, epigen, epiregulin, and amphiregulin, which typically bind to ErbB1 and induce heterodimerization of ErbB1 with ErbB3.

The term “bispecific antibody” as used herein refers to a protein comprising two antigen-binding sites, a first binding site exhibiting immunospecific binding to a first antigen or epitope and a second binding site exhibiting immunospecific binding to a second antigen or epitope distinct from the first. An anti-ErbB3/anti-ErbB2 bispecific antibody is an antibody that comprises two binding sites, one that immunospecifically binds to the ectodomain of ErbB3 and another that immunospecifically binds to the ectodomain of ErbB2.

As used herein, the term “EGFR inhibitor” or “inhibitor of EGFR signaling” is intended to include therapeutic agents that inhibit, downmodulate, suppress or downregulate EGFR signaling activity. The term is intended to include chemical compounds, such as small molecule inhibitors (e.g., small molecule tyrosine kinase inhibitors) and biologic agents, such as antibodies, interfering RNA (shRNA, siRNA), soluble receptors and the like.

As used herein, the term “VEGF inhibitor” is intended to include therapeutic agents that inhibit, downmodulate, suppress or downregulate VEGF signaling activity. The term is intended to include chemical compounds, such as small molecule inhibitors (e.g., small molecule tyrosine kinase inhibitors) and biologic agents, such as antibodies, interfering RNA (shRNA, siRNA), soluble receptors and the like.

As used herein, the term “mTOR inhibitor” is intended to include therapeutic agents that inhibit, downmodulate, suppress or downregulate mammalian target of rapamycin (mTOR). The term is intended to include chemical compounds, such as small molecule inhibitors (e.g., small molecule serine/threonine kinase inhibitors) and biologic agents, such as antibodies, interfering RNA (shRNA, siRNA), soluble receptors and the like.

As used herein, the term “MEK inhibitor” is intended to include therapeutic agents that inhibit, downmodulate, suppress or downregulate mitogen activated protein kinase kinase (MEK). The term is intended to include chemical compounds, such as small molecule inhibitors (e.g., small molecule serine/threonine kinase inhibitors) and biologic agents, such as antibodies, interfering RNA (shRNA, siRNA), soluble receptors and the like.

As used herein, the term “PI3K inhibitor” is intended to include therapeutic agents that inhibit, downmodulate, suppress or downregulate phosphatidylinositol-3-kinase (PI3K). The term is intended to include chemical compounds, such as small molecule inhibitors (e.g., small molecule serine/threonine kinase inhibitors) and biologic agents, such as antibodies, interfering RNA (shRNA, siRNA), soluble receptors and the like.

The terms “suppress”, “suppression”, “inhibit” and “inhibition” as used interchangeably herein, refer to any statistically significant decrease in biological activity (e.g., tumor cell growth), including full blocking of the activity. For example, “inhibition” can refer to a decrease of about 10%, 20%, 30%, 40%, 50%, 60%, 70%, 80%, 90%, or 100% in biological activity.

The term “patient” includes a human or other mammalian animal that receives either prophylactic or therapeutic treatment.

The terms “treat,” “treating,” and “treatment,” as used herein, refer to therapeutic or preventative measures described herein. The methods of “treatment” employ administration to a patient of an ErbB3 inhibitor such as those described herein, for example, a patient having a hormone refractory breast cancer tumor, in order to cure, delay, reduce the severity of, or ameliorate one or more symptoms of the disease or disorder or recurring disease or disorder, or in order to prolong the survival of a patient beyond that expected in the absence of such treatment.

The term “effective amount,” as used herein, refers to that amount of an agent, such as an ErbB3 inhibitor, e.g., an anti-ErbB3 antibody, which is sufficient to effect treatment, prognosis or diagnosis of a hormone refractory breast cancer, when administered to a patient. A therapeutically effective amount will vary depending upon the patient and disease condition being treated, the weight and age of the patient, the severity of the disease condition, the manner of administration and the like, which can readily be determined by one of ordinary skill in the art. The dosages for administration can range from, for example, about 1 ng to about 10,000 mg, about 5 ng to about 9,500 mg, about 10 ng to about 9,000 mg, about 20 ng to about 8,500 mg, about 30 ng to about 7,500 mg, about 40 ng to about 7,000 mg, about 50 ng to about 6,500 mg, about 100 ng to about 6,000 mg, about 200 ng to about 5,500 mg, about 300 ng to about 5,000 mg, about 400 ng to about 4,500 mg, about 500 ng to about 4,000 mg, about 1 μg to about 3,500 mg, about 5 μg to about 3,000 mg, about 10 μg to about 2,600 mg, about 20 μg to about 2,575 mg, about 30 μg to about 2,550 mg, about 40 μg to about 2,500 mg, about 50 μg to about 2,475 mg, about 100 μg to about 2,450 mg, about 200 μg to about 2,425 mg, about 300 μg to about 2,000, about 400 μg to about 1,175 mg, about 500 μg to about 1,150 mg, about 0.5 mg to about 1,125 mg, about 1 mg to about 1,100 mg, about 1.25 mg to about 1,075 mg, about 1.5 mg to about 1,050 mg, about 2.0 mg to about 1,025 mg, about 2.5 mg to about 1,000 mg, about 3.0 mg to about 975 mg, about 3.5 mg to about 950 mg, about 4.0 mg to about 925 mg, about 4.5 mg to about 900 mg, about 5 mg to about 875 mg, about 10 mg to about 850 mg, about 20 mg to about 825 mg, about 30 mg to about 800 mg, about 40 mg to about 775 mg, about 50 mg to about 750 mg, about 100 mg to about 725 mg, about 200 mg to about 700 mg, about 300 mg to about 675 mg, about 400 mg to about 650 mg, about 500 mg, or about 525 mg to about 625 mg, of an antibody or antigen binding portion thereof, as provided herein. Dosing may be, e.g., every week, every 10 days, every 2 weeks, every 18 days, every three weeks, every 4 weeks, every 5 weeks or every 6 weeks. Dosage regimens may be adjusted to provide the optimum therapeutic response. An effective amount is also one in which any toxic or detrimental effects (side effects) of the ErbB3 inhibitor are minimized and/or outweighed by the beneficial effects. For MM-121, administration may be intravenous at exactly or about 6 mg/kg or 12 mg/kg weekly, or 12 mg/kg or 24 mg/kg biweekly. For MM-111, dosing may be intravenous at exactly or about every x days with an initial loading dose of exactly or about y mg/kg and subsequent maintenance doses of exactly or about z mg/kg, where x, y and z are: 7, 25, and 20, or 10, 40 and 30, or 14, 60, and 44, or 18, 90, and 75, or 21, 120, and 105. Additional preferred dosing regimens are described below.

The terms “anti-cancer agent” and “antineoplastic agent” refer to drugs used to treat malignancies, such as cancerous growths. Drug therapy may be used alone, or in combination with other treatments such as surgery or radiation therapy.

“Therapeutic synergy” refers to a phenomenon where treatment of patients with a combination of therapeutic agents manifests a therapeutically superior outcome to the outcome achieved by each individual constituent of the combination used at its optimum dose (T. H. Corbett et al., 1982, Cancer Treatment Reports, 66, 1187). In this context a therapeutically superior outcome is one in which the patients either a) exhibit fewer incidences of adverse events while receiving a therapeutic benefit that is equal to or greater than that where individual constituents of the combination are each administered as monotherapy at the same dose as in the combination, or b) do not exhibit dose-limiting toxicities while receiving a therapeutic benefit that is greater than that of treatment with each individual constituent of the combination when each constituent is administered in at the same doses in the combination(s) as is administered as individual components. In xenograft models, a combination, used at its maximum tolerated dose, in which each of the constituents will be present at a dose generally not exceeding its individual maximum tolerated dose, manifests therapeutic synergy when decrease in tumor growth achieved by administration of the combination is greater than the value of the decrease in tumor growth of the best constituent when the constituent is administered alone.

Thus, in combination, the components of such combinations have an additive or superadditive effect on suppressing pancreatic tumor growth, as compared to monotherapy with the anti-ErbB3 antibody or treatment with the chemotherapeutic(s) in the absence of antibody therapy. By “additive” is meant a result that is greater in extent (e.g., in the degree of reduction of tumor mitotic index or of tumor growth or in the degree of tumor shrinkage or the frequency and/or duration of symptom-free or symptom-reduced periods) than the best separate result achieved by monotherapy with each individual component, while “superadditive” is used to indicate a result that exceeds in extent the sum of such separate results. In one embodiment, the additive effect is measured as slowing or stopping of pancreatic tumor growth. The additive effect can also be measured as, e.g., reduction in size of a pancreatic tumor, reduction of tumor mitotic index, reduction in number of metastatic lesions over time, increase in overall response rate, or increase in median or overall survival.

One non-limiting example of a measure by which effectiveness of a therapeutic treatment can be quantified is by calculating the log 10 cell kill, which is determined according to the following equation:

log 10 cell kill=TC(days)/3.32×Td

in which T C represents the delay in growth of the cells, which is the average time, in days, for the tumors of the treated group (T) and the tumors of the control group (C) to have reached a predetermined value (1 g, or 10 mL, for example), and Td represents the time, in days necessary for the volume of the tumor to double in the control animals. When applying this measure, a product is considered to be active if log 10 cell kill is greater than or equal to 0.7 and a product is considered to be very active if log 10 cell kill is greater than 2.8. Using this measure, a combination, used at its own maximum tolerated dose, in which each of the constituents is present at a dose generally less than or equal to its maximum tolerated dose, exhibits therapeutic synergy when the log 10 cell kill is greater than the value of the log 10 cell kill of the best constituent when it is administered alone. In an exemplary case, the log 10 cell kill of the combination exceeds the value of the log 10 cell kill of the best constituent of the combination by at least 0.1 log cell kill, at least 0.5 log cell kill, or at least 1.0 log cell kill. Various aspects and embodiments are described in further detail in the following subsections.

I. ErbB3 Inhibitors

As described in further detail herein, the methods and compositions provided herein involve the use of one or more ErbB3 inhibitors.

In one embodiment, the ErbB3 inhibitor is an anti-ErbB3 antibody, e.g., a monoclonal antibody. Useful anti-ErbB3 antibodies (or VH/VL domains derived therefrom) can be made using methods well known in the art. Alternatively, art recognized anti-ErbB3 antibodies can be used. For example, Ab#3, Ab #14, Ab #17, Ab #19, described in U.S. Pat. No. 7,846,440, can be used. Antibodies that compete with any of these antibodies for binding to ErbB3 also can be used. Additional art-recognized anti-ErbB3 antibodies which can be used include those disclosed in U.S. Pat. No. 7,285,649; US20200310557; US20100255010, as well as antibodies IB4C3 and 2D1D12 (U3 Pharma Ag), both of which are described in e.g., US20040197332 and are produced by hybridoma cell lines DSM ACC 2527 or DSM ACC 2517 (deposited at DSMZ); anti-ErbB3 antibody referred to as AMG888 (U3-1287-U3 Pharma Ag and Amgen) described in U.S. Pat. No. 7,705,130; and monoclonal antibody 8B8 (ATCC® HB-12070™), described in U.S. Pat. No. 5,968,511, and the anti-ErbB3 antibody referred to as AV-203 (Aveo Pharmaceuticals) which is described in US patent publication No. 20110256154. Other useful anti-ErbB3 antibodies are disclosed in the art in the context of a bispecific antibody (see e.g., B2B3-1 or B2B3-2 in WO/2009126920 and those described in U.S. Pat. No. 7,846,440, US 20090291085, US 20100056761, and US 20100266584. An exemplary anti-ErbB3 monoclonal antibody comprises MM-121, a fully human anti-ErbB3 antibody currently undergoing Phase II clinical trials. MM-121 is described further in PCT Publication No. WO 2008/100624 and U.S. Pat. No. 7,846,440, and comprises V_{H} and V_{L} sequences as shown in SEQ ID NOs: 1 and 2, respectively. MM-121 is referred to as “Ab #6” in U.S. Pat. No. 7,846,440. Alternately, the anti-ErbB3 monoclonal antibody is an antibody that competes with MM-121 for binding to ErbB3. In another embodiment, the anti-ErbB3 antibody is an antibody comprising the V_{H} and V_{L} CDR sequences of MM-121, which are shown in SEQ ID NOs: 3-5 (V_{H} CDR1, 2, 3) and 6-8 (V_{L} CDR1, 2, 3), respectively. In another embodiment, the anti-ErbB3 antibody has heavy and light chains comprising the amino acid sequences set forth in SEQ ID NOs 42 and 43, respectively. Other examples of anti-ErbB3 antibodies include Ab #3, Ab #14, Ab #17 and Ab #19, also described further in WO 2008/100624 and having V_{H} and V_{L} sequences as shown in SEQ ID NOs: 9 and 10, 17 and 18, 25 and 26, and 33 and 34 respectively. In another embodiment, the anti-ErbB3 antibody is an antibody comprising the V_{H} and V_{L} CDR sequences of Ab #3 (shown in SEQ ID NOs: 11-13 and 14-18, respectively) or antibody comprising the V_{H} and V_{L} CDR sequences of Ab #14 (shown in SEQ ID NOs: 19-21 and 22-24, respectively) or an antibody comprising the V_{H} and V_{L} CDR sequences of Ab #17 (shown in SEQ ID NOs: 27-29 and 30-32, respectively) or an antibody comprising the V_{H} and V_{L} CDR sequences of Ab #19 (shown in SEQ ID NOs: 35-37 and 38-40, respectively).

Alternately, the anti-ErbB3 antibody is a monoclonal antibody or antigen binding portion thereof which binds an epitope of human ErbB3 comprising residues 92-104 of SEQ ID NO:41 and is characterized by inhibition of proliferation of a cancer cell expressing ErbB3. The cancer cell may be a MALME-3M cell, an AdrR cell, or an ACHN cell and the proliferation may be reduced by at least 10% relative to control. In an additional embodiment this isolated monoclonal antibody or antigen binding portion thereof binds an epitope comprising residues 92-104 and 129 of SEQ ID NO:41.

In yet another embodiment, the anti-ErbB3 antibody can comprise a mixture, or cocktail, of two or more anti-ErbB3 antibodies, each of which binds to a different epitope on ErbB3. In one embodiment, the mixture, or cocktail, comprises three anti-ErbB3 antibodies, each of which binds to a different epitope on ErbB3.

In another embodiment, the ErbB3 inhibitor comprises a nucleic acid molecule, such as an RNA molecule, that inhibits the expression or activity of ErbB3. RNA antagonists of ErbB3 have been described in the art (see e.g., U.S. Patent Application Publication No. 20080318894). Moreover, interfering RNAs specific for ErbB3, such as shRNAs or siRNAs that specifically inhibits the expression and/or activity of ErbB3, have been described in the art.

In yet another embodiment, the ErbB3 inhibitor comprises a soluble form of ErbB3 that inhibits signaling through the ErbB3 pathway. Such soluble ErbB3 molecules have been described in the art (see e.g., U.S. Pat. No. 7,390,632, U.S. Pat. No. 7,638,303 and U.S. Pat. No. 7,638,302, each by Maihle et al., and U.S. Pat. No. 7,919,098 by Zhou).

II. Anti-ErbB2 Antibodies

The methods and compositions provided herein may involve the use of one or more anti-ErbB2 antibodies that can inhibit the binding of heregulin to ErbB2/ErbB3 heterodimers. Suitable anti-ErbB2 antibodies include C6.5 (and the numerous derivatives thereof) described in U.S. Pat. No. 5,977,322, as well as trastuzumab, as described in U.S. Pat. No. 6,054,297, or pertuzumab, as described in U.S. Pat. No. 6,949,245.

III. Bispecific Antibodies

The methods and compositions provided herein may involve the use of one or more bispecific antibodies, preferably ones that can inhibit the binding of heregulin to ErbB2/ErbB3 heterodimers. Such bispecific antibodies include ALM, as described in U.S. Pat. No. 7,332,580, as well as A5-HSA-ML3.9, A5-HSA-B1D2, B12-HSA-B1D2, A5-HSA-F5B6H2, H3-HSA-F5B6H2, F4-HSA-F5B6H2, and H3-HSA-B1D2, as described in U.S. Patent Application Publication No. 20110059076, and PCT publication number WO2009/126920, each of which, as described therein, have variant forms such as those comprising mHSA. In one embodiment, the bispecific antibody comprises SEQ ID NO:44.

IV. Methods

In one aspect, use of an ErbB3 inhibitor for the manufacture of a medicament for the treatment of hormone refractory breast cancer is provided, in certain embodiments the breast cancer is estrogen receptor positive hormone refractory breast cancer.

In another aspect, a method of suppressing growth of a hormone refractory breast cancer cell (optionally an ER+ hormone refractory breast cancer cell) is provided, the method comprising contacting the cell with an effective amount of an ErbB3 inhibitor.

In another aspect, a method of suppressing growth of a hormone refractory breast cancer tumor (optionally an ER+ hormone refractory breast cancer tumor) in a patient is provided, the method comprising administering to the patient an effective amount of an ErbB3 inhibitor.

In still another aspect, a method of treating a breast cancer tumor (optionally an estrogen receptor positive hormone refractory breast cancer tumor) in a patient is provided, the method comprising:

selecting a patient with a hormone refractory breast cancer tumor; and

administering to the patient an effective amount of an ErbB3 inhibitor.

In another aspect, the patient with a hormone refractory breast cancer tumor is a patient further selected by use of the selection methods disclosed in pending international application PCT/US2009/054051.

The hormone refractory breast cancer to be treated with ErbB3 inhibitor may co-express ErbB1 (EGFR), ErbB3, and heregulin (HRG). Expression of EGFR and HRG can be identified by RT-PCR or by standard immunoassay techniques, such as ELISA assay, immunohistochemical staining of formalin-fixed, paraffin-embedded tissues (e.g., breast cancer tissues routinely processed for histological evaluation), using an anti-EGFR antibody, anti-ErbB3 antibody or an anti-HRG antibody. Additional characteristics of preferred tumors for treatment in accordance with the disclosure herein are set forth in pending U.S. Patent Publication No. 20110027291, which claims priority to PCT application No. PCT/US2009/054051.

In one embodiment, the ErbB3 inhibitor administered to the patient is an anti-ErbB3 antibody. An exemplary anti-ErbB3 antibody is MM-121, comprising V_{H} and V_{L} sequences as shown in SEQ ID NOs: 1 and 2, respectively, or an antibody comprising V_{H} CDR1, 2 and 3 sequences as shown in SEQ ID NOs: 3-5, respectively, and V_{L} CDR1, 2 and 3 sequences as shown in SEQ ID NOs: 6-8, respectively (i.e., the V_{H} and V_{L} CDRs of MM-121). Additional non-limiting exemplary anti-ErbB3 antibodies and other forms of ErbB3 inhibitors are described in detail in Subsection I above.

The ErbB3 inhibitor can be administered to the patient by any route suitable for the effective delivery of the inhibitor to the patient. For example, many small molecule inhibitors are suitable for oral administration. Antibodies and other biologic agents typically are administered parenterally, e.g., intravenously, intraperitoneally, subcutaneously or intramuscularly. Various routes of administration, dosages and pharmaceutical formulations suitable for use in the methods provided herein are described in further detail below.

In further aspects, the methods described herein include methods inhibition (e.g., at least partial blockade) of heregulin-mediated activating phosphorylation of estrogen receptors. These methods involve the use of one or more antibodies that can inhibit the binding of heregulin to ErbB2/ErbB3 heterodimers to inhibit such phosphorylation. In certain embodiments, such methods further include optional co-administration of hormone modulatory drugs, including estrogen receptor antagonists and aromatase inhibitors.

V. Pharmaceutical Compositions

In another aspect, pharmaceutical compositions are provided that can be used in the methods disclosed herein, i.e., pharmaceutical compositions for treating hormone refractory breast cancer tumors.

In one embodiment, the pharmaceutical composition for treating hormone refractory breast cancer comprises an ErbB3 inhibitor and a pharmaceutical carrier. The ErbB3 inhibitor can be formulated with the pharmaceutical carrier into a pharmaceutical composition. Additionally, the pharmaceutical composition can include, for example, instructions for use of the composition for the treatment of patients for hormone refractory breast cancer tumors.

In one embodiment, the ErbB3 inhibitor in the composition is an anti-ErbB3 antibody, e.g., MM-121 or an antibody comprising the V_{H} and V_{L} CDRs of MM-121 positioned in the antibody in the same relative order as they are present in MM-121 so as to provide immunospecific binding of ErbB3. Additional non-limiting exemplary anti-ErbB3 antibodies and other forms of ErbB3 inhibitors are described in detail in Subsection I above.

As used herein, “pharmaceutically acceptable carrier” includes any and all solvents, dispersion media, coatings, antibacterial and antifungal agents, isotonic and absorption delaying agents, buffers, and other excipients that are physiologically compatible. Preferably, the carrier is suitable for parenteral, oral, or topical administration. Depending on the route of administration, the active compound, e.g., small molecule or biologic agent, may be coated in a material to protect the compound from the action of acids and other natural conditions that may inactivate the compound.

Pharmaceutically acceptable carriers include sterile aqueous solutions or dispersions and sterile powders for the extemporaneous preparation of sterile injectable solutions or dispersion, as well as conventional excipients for the preparation of tablets, pills, capsules and the like. The use of such media and agents for the formulation of pharmaceutically active substances is known in the art. Except insofar as any conventional media or agent is incompatible with the active compound, use thereof in the pharmaceutical compositions provided herein is contemplated. Supplementary active compounds can also be incorporated into the compositions.

A pharmaceutically acceptable carrier can include a pharmaceutically acceptable antioxidant. Examples of pharmaceutically-acceptable antioxidants include: (1) water soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal chelating agents, such as citric acid, ethylenediamine tetraacetic acid (EDTA), sorbitol, tartaric acid, phosphoric acid, and the like.

Examples of suitable aqueous and nonaqueous carriers which may be employed in the pharmaceutical compositions provided herein include water, ethanol, polyols (such as glycerol, propylene glycol, polyethylene glycol, and the like), and suitable mixtures thereof, and injectable organic esters, such as ethyl oleate. When required, proper fluidity can be maintained, for example, by the use of coating materials, such as lecithin, by the maintenance of the required particle size in the case of dispersions, and by the use of surfactants. In many cases, it will be preferable to include isotonic agents, for example, sugars, polyalcohols such as mannitol, sorbitol, or sodium chloride in the composition. Prolonged absorption of the injectable compositions can be brought about by including in the composition an agent that delays absorption, for example, monostearate salts and gelatin.

These compositions may also contain functional excipients such as preservatives, wetting agents, emulsifying agents and dispersing agents.

Therapeutic compositions typically must be sterile, non-pyrogenic, and stable under the conditions of manufacture and storage. The composition can be formulated as a solution, microemulsion, liposome, or other ordered structure suitable to high drug concentration.

Sterile injectable solutions can be prepared by incorporating the active compound in the required amount in an appropriate solvent with one or a combination of ingredients enumerated above, as required, followed by sterilization, e.g., by microfiltration. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying (lyophilization) that yield a powder of the active ingredient plus any additional desired ingredient from a previously sterile-filtered solution thereof. The active agent(s) may be mixed under sterile conditions with additional pharmaceutically acceptable carrier(s), and with any preservatives, buffers, or propellants which may be required.

Prevention of presence of microorganisms may be ensured both by sterilization procedures, supra, and by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Pharmaceutical compositions comprising an ErbB3 inhibitor can be administered alone or in combination therapy. For example, the combination therapy can include a composition provided herein comprising an ErbB3 inhibitor and at least one or more additional therapeutic agents, such as one or more chemotherapeutic agents known in the art, discussed in further detail in Subsection IV below. Pharmaceutical compositions can also be administered in conjunction with radiation therapy and/or surgery.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation.

Exemplary dosage ranges for administration of an antibody include: 10-1000 mg (antibody)/kg (body weight of the patient), 10-800 mg/kg, 10-600 mg/kg, 10-400 mg/kg, 10-200 mg/kg, 30-1000 mg/kg, 30-800 mg/kg, 30-600 mg/kg, 30-400 mg/kg, 30-200 mg/kg, 50-1000 mg/kg, 50-800 mg/kg, 50-600 mg/kg, 50-400 mg/kg, 50-200 mg/kg, 100-1000 mg/kg, 100-900 mg/kg, 100-800 mg/kg, 100-700 mg/kg, 100-600 mg/kg, 100-500 mg/kg, 100-400 mg/kg, 100-300 mg/kg and 100-200 mg/kg. Exemplary dosage schedules include once every three days, once every five days, once every seven days (i.e., once a week), once every 10 days, once every 14 days (i.e., once every two weeks), once every 21 days (i.e., once every three weeks), once every 28 days (i.e., once every four weeks) and once a month.

It may be advantageous to formulate parenteral compositions in unit dosage form for ease of administration and uniformity of dosage. Unit dosage form as used herein refers to physically discrete units suited as unitary dosages for the patients to be treated; each unit contains a predetermined quantity of active agent calculated to produce the desired therapeutic effect in association with any required pharmaceutical carrier. The specification for unit dosage forms are dictated by and directly dependent on (a) the unique characteristics of the active compound and the particular therapeutic effect to be achieved, and (b) the limitations inherent in the art of compounding such an active compound for the treatment of sensitivity in individuals.

Actual dosage levels of the active ingredients in the pharmaceutical compositions disclosed herein may be varied so as to obtain an amount of the active ingredient which is effective to achieve the desired therapeutic response for a particular patient, composition, and mode of administration, without being toxic to the patient. “Parenteral” as used herein in the context of administration means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion.

The phrases “parenteral administration” and “administered parenterally” as used herein refer to modes of administration other than enteral (i.e., via the digestive tract) and topical administration, usually by injection or infusion, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Intravenous injection and infusion are often (but not exclusively) used for antibody administration.

When agents provided herein are administered as pharmaceuticals, to humans or animals, they can be given alone or as a pharmaceutical composition containing, for example, 0.001 to 90% (more preferably, 0.005 to 70%, such as 0.01 to 30%) of active ingredient in combination with a pharmaceutically acceptable carrier.

VI. Combination Therapy

In certain embodiments, the methods and uses provided herein for suppressing growth of hormone refractory breast cancer cells or for treating a patient with a hormone refractory breast tumor or can comprise administration of an ErbB3 inhibitor and at least one additional anti-cancer agent that is not an ErbB3 inhibitor.

In one embodiment, the at least one additional anti-cancer agent comprises at least one chemotherapeutic drug. Non-limiting examples of such chemotherapeutic drugs include platinum-based chemotherapy drugs (e.g., cisplatin, carboplatin), taxanes (e.g., paclitaxel (Taxol®), docetaxel (Taxotere®), EndoTAG-1™ (a formulation of paclitaxel encapsulated in positively charged lipid-based complexes; MediGene®), Abraxane® (a formulation of paclitaxel bound to albumin), tyrosine kinase inhibitors (e.g., imatinib/Gleevec®, sunitinib/Sutent®, dasatinib/Sprycel®), and combinations thereof.

In another embodiment, the at least one additional anti-cancer agent comprises an EGFR inhibitor, such as an anti-EGFR antibody or a small molecule inhibitor of EGFR signaling. An exemplary anti-EGFR antibody is cetuximab (Erbitux®, ImClone Systems). Other examples of anti-EGFR antibodies include MM-151 (further described in Bukhalid et al., copending commonly assigned U.S. Patent Application Ser. No. 61/504,633, filed on Jul. 5, 2011), Sym004 (Symphogen, Pederson et al., Cancer Research Jan. 15, 2010 70; 588, also see U.S. Pat. No. 7,887,805), matuzumab (EMD72000), panitumumab (Vectibix®; Amgen); nimotuzumab (TheraCIM™) and mAb 806. An exemplary small molecule inhibitor of the EGFR signaling pathway is gefitinib (Iressa®), which is commercially available from AstraZeneca and Teva. Other examples of small molecule inhibitors of EGFR signaling include erlotinib HCL (OSI-774; Tarceva®; OSI Pharma), lapatinib (Tykerb®, GlaxoSmithKline), canertinib (canertinib dihydrochloride, Pfizer), pelitinib (Pfizer); PKI-166 (Novartis); PD158780; afatinib (Tomtovok®, Boehringer Ingleheim); and AG 1478 (4-(3-Chloroanillino)-6,7-dimethoxyquinazoline).

In yet another embodiment, the at least one additional anti-cancer agent comprises a VEGF inhibitor. An exemplary VEGF inhibitor comprises an anti-VEGF antibody, such as bevacizumab (Avastatin®; Genentech).

In another embodiment, the at least one additional anti-cancer agent comprises an IGF1R inhibitor, such as an anti-EGFR antibody or a small molecule inhibitor of EGFR signaling. Examples of anti-IGF1R inhibitors include dalotuzumab (Merck, also MK-0646), AMG-479 (Amgen), R1507 (Roche), figitumumab (Pfizer), IMC-A12 (Imclone/Lilly), and MM-141, a bispecific ErbB3/IGF1R inhibitor (further described in Lugovskoy et al., copending commonly assigned U.S. Patent Application Ser. No. 61/558,192, filed Nov. 10, 2011). Examples of small molecule IGF1R inhibitors include XL228 (Exelixis) and BMS-754807 (BMS).

### EXAMPLES

### Example 1

### MM-121 Treatment of ER+, Hormone Refractory Mammary Tumors

Analyses of the anti-tumor efficacy and tolerability of MM-121 treatment of ER+ hormone refractory mammary tumor-bearing mice are carried out using xenografts of tamoxifen-resistant variants of MCF7 human mammary carcinoma cells. Tamoxifen-resistant human mammary carcinoma cell lines TAMR-1, TAMR-7, and TAMR-8 cells are obtained from the laboratory of A. E. Lykkesfeldt (Department of Tumor Endocrinology, Division for Cancer Biology, Danish Cancer Society. Strandboulevarden 49, DK-2100 Copenhagen 0, Denmark). These are grown as xenografts in female athymic nu+/nu+ nude mice obtained from Charles River Laboratories International. SCID mice (C.B.-17/IcrACCscid) obtained from the Arizona Cancer Center breeding colony, Tucson, Ariz., are also suitable. The mice are housed in Tecniplast® Individually Ventilated polycarbonate (Makrolon®) Cages (IVC) set in climate-controlled rooms and have free access to food and acidified water. Mice are injected under general anesthesia with ˜(about) 10⁷ TAMR-1, TAMR-7, or TAMR-8 cells either subcutaneously in the flank or into the mammary fat pad.

To investigate anti-tumor efficacy in monotherapy, MM-121 or vehicle control (100 μL) is given to tumor-bearing mice (i.e., after 14 days of tumor growth following injection of cells) at 600 μg per mouse (MM-121 as a 6 mg/mL solution in PBS) by IP injection every three days. Control mice receive the PBS vehicle only. Efficacy is determined by comparing tumor growth between the antibody-treated mice and the vehicle control mice and is expressed as the experimental to control ratio of median relative tumor volumes (T/C value). A minimum T/C value below 50% is a prerequisite for rating a treatment as effective. The control and experimental groups each contain 10 mice bearing one tumor each. To obtain 30 mice bearing tumors of similar sizes for randomization, 40 mice per tumor are implanted unilaterally.

Mice are randomized and therapy begins when a sufficient number of individual tumors have grown to a volume of approximately 200 mm³. Tumors are measured (L x W) by digital caliper measurement and the tumor volume is calculated using the formula π/6 (W²×L). The first dose is administered either on Day 0 (day of randomization) or one day later.

Approximately 24 hours after administration of the final dose all mice are bled to prepare serum; in addition, tumors are collected from the same mice for flash-freezing and paraffin embedding (FFPE) (½ tumor each).

According to regulations for animal experiments, mice are sacrificed if the tumor volume exceeds 1800 mm³ (one tumor per mouse). Mice are monitored and dosed until their tumors have grown to that size but no longer than 60 days. Thereafter, they are sacrificed for sample collection.

At the end of the study, approximately 24 hours after administration of the final dose, all mice on study are bled sublingually to obtain a maximum amount of blood for the preparation of serum. Serum is aliquoted in 2 tubes with approximately 250 μL in each.

In addition, tumors from all mice are excised without delay for snap-freezing in liquid nitrogen (½ tumor, COVARIS bags for the storage of samples are provided) and for fixation in 10% buffered formalin for <24 hours, subsequent dehydration and paraffin embedding (½ tumor).

Animal weights and tumor diameters (W and L) are measured twice weekly and tumor volumes are calculated using the formula π/6 (W²×L). Tumor growth curves are plotted. Tumor inhibition and absolute growth delay for 2 and 4 doubling times are calculated.

Results of experiments carried out substantially as described will demonstrate that MM-121 treatment inhibits or stops tumor growth, and in some cases reduces tumor size.

### Example 2

### MM-121 Inhibition of HRG-Induced ER Phosphorylation In Vitro

MCF7 cells are either untreated or pretreated with MM-121 (250 nM) for 1 hour. Cells are then stimulated with heregulin betal (EGF domain, 10 nM R&D systems), betacellulin (20 nM, R&D systems) or estrogen (beta estradiol—100 nM, Sigma) for 30 minutes, or left unstimulated. Lysates of the cells are analyzed by western blot probed for pER and for pErbB3.

To demonstrate the ability of MM-121 to reduce heregulin-induced activation of the estrogen receptor, treatments were tested in the ER⁺, PR⁺, ErbB2⁺ cancer cell line MCF7 using the methods described above or trivial variations thereof. Cells were either untreated or pretreated with MM-121. Untreated and pretreated cells were stimulated with heregulin, betacellulin, or estrogen. Cell lysates were analyzed by western blot for phosphorylated forms of ErbB3 and estrogen receptor.

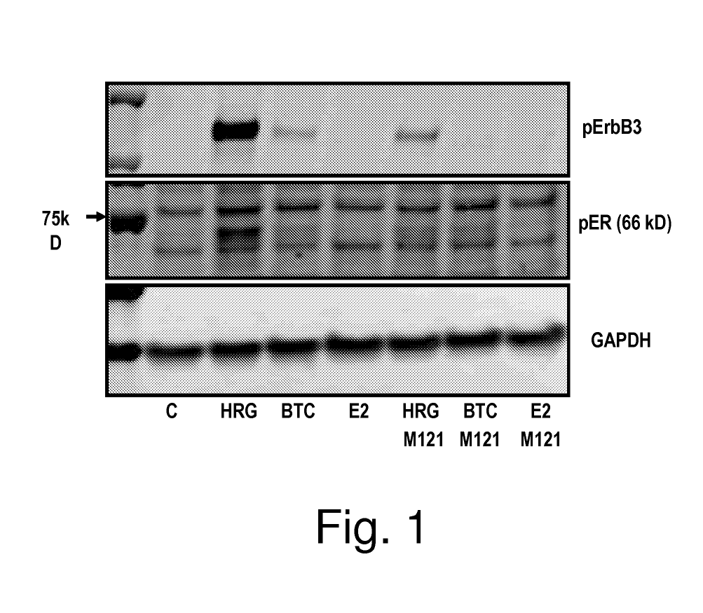

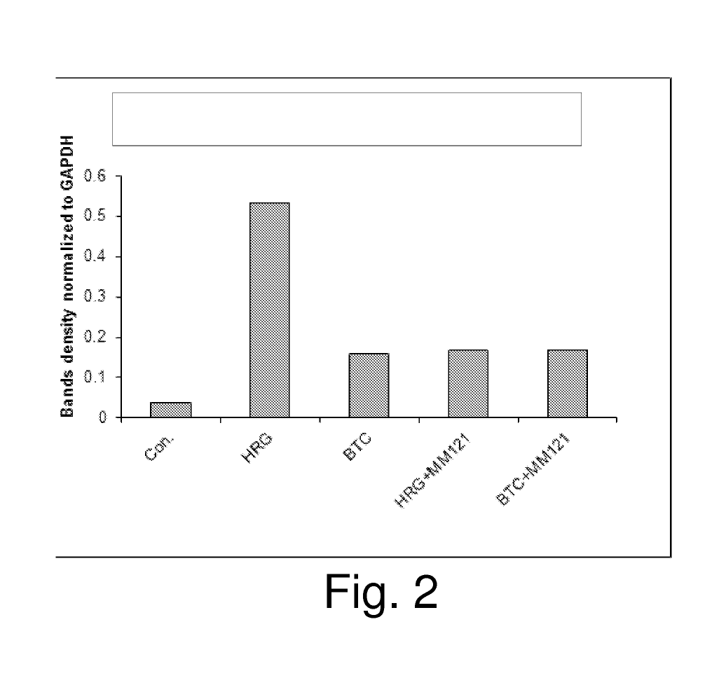

As shown in FIG. 1 (western blot) and FIG. 2 (densitometry of the data in FIG. 1), untreated heregulin-stimulated cells, and to a lesser extent (about ⅔ less) untreated betacellulin-stimulated cells, exhibited phosphorylation of both the estrogen receptor and ErbB3. In contrast, heregulin-stimulated cells pretreated with MM-121 exhibited a substantial (about ⅔) reduction in the amount of pErbB3 and pER. The results demonstrate that heregulin-induced activation of the estrogen receptor is mediated by ErbB3 and that MM-121 can inhibit this mode of estrogen receptor activation. Surprisingly, heregulin stimulation induced a dramatically (at least four-fold) higher level of ER phosphorylation than did estrogen.

### Example 3

### Restoring Sensitivity and/or Preventing Resistance to Aromatase Inhibitors by Co-Administration with MM-121

Aromatase inhibitor (AI) treatment is well tolerated by patients, and the therapy is effective for a relatively long period. However, patients who are initially responsive to AI treatment can become resistant to the drug. To investigate the mechanism of AI resistance, a xenograft model was developed that corresponds to ER+ postmenopausal breast cancer. Tumors for this intratumoral aromatase xenograft model are grown from MCF7 human breast adenocarcinoma cells that have been stably transfected with a human placental aromatase gene to provide a non-ovarian source of estrogen production in ovariectomized athymic mice (MCF-7CA, see e.g. Brodie et al., Clinical Cancer Research 884s Vol. 11, 884s-888s, Jan. 15, 2005 (Suppl.)). Sufficient estrogen is produced (from aromatization of injected androstenedione) by the MCF7-CA cells to stimulate their proliferation and tumor formation. This is a model of a postmenopausal breast cancer patient with tumors that express aromatase and are free of gonadotropin feedback regulation.

MCF-7CA Tumors:

MCF-7CA cells were cultured in Eagle's minimum essential medium containing 5% fetal bovine serum and neomycin. The culture medium was changed twice weekly. Subconfluent MCF-7CA cells were scraped into Hank's solution and centrifuged at 1,000 rpm for 2 min at 4° C. The cells were then resuspended in Matrigel™ (10 mg/ml) to make a cell suspension of 2-5×10⁷ cells/ml. Ovariectomized female BALB/c athymic mice 4-6 weeks of age (20-22 g body weight) were housed in a pathogen-free environment under controlled conditions of light and humidity and received food and water ad libitum. Each mouse was inoculated subcutaneously (s.c.) with 0.1 ml of the cell suspension. Animals were then injected s.c. daily with 0.1 mg androstenedione/mouse. Growth rates were determined by measuring the tumors with calipers weekly. Tumor volumes were calculated according to the formula for a sphere (4/3 r12. r 2). When tumors reached a measurable size, mice were divided into groups of 10 animals with equivalent tumor volumes (300 mm³). Mice were treated with 0.1 ml compounds in 0.3% hydroxypropylcellulose (HPC) for 6 weeks.

Letrozole Vs. Letrozole+MM121 in Letrozole Resistant Model

To demonstrate the efficacy of MM-121 in combination with letrozole in a letrozole-resistant xenograft model, tumor bearing mice were prepared as described above and randomized into 3 groups of 10 mice each and one group of 30 mice, each containing mice with a similar size distribution of tumors. For initial treatment during the period when development of resistance was expected, one group was treated with PBS, Q3D, i.p. as a control; one group was treated with MM-121 alone (600 μg MM121 in 0.2 ml PBS/mouse every 3 days i.p.); the 30-mouse group a was treated with letrozole alone (10 μg/mouse/day); and a final group was treated with both letrozole and MM-121. Tumors were measured weekly and tumor volume was calculated. Mice were sacrificed if tumors continued to grow to volumes of greater than about 1400-1700 mm³. When the tumors in the group of mice treated with letrozole alone became resistant and exceeded about 600 mm³ in volume, the group was subdivided into 3 groups of ten mice each. To investigate the effect of MM-121 combination therapy on these resistant tumors, treatment of these three groups continued as follows: one group continued to receive letrozole alone (10 μg/mouse/day), one group stopped receiving daily letrozole and was treated with MM-121 (600 μg Q3D, i.p.) alone, and one group was treated with a combination of letrozole (10 μg/mouse/day) and MM-121 (600 μg Q3D, i.p.).

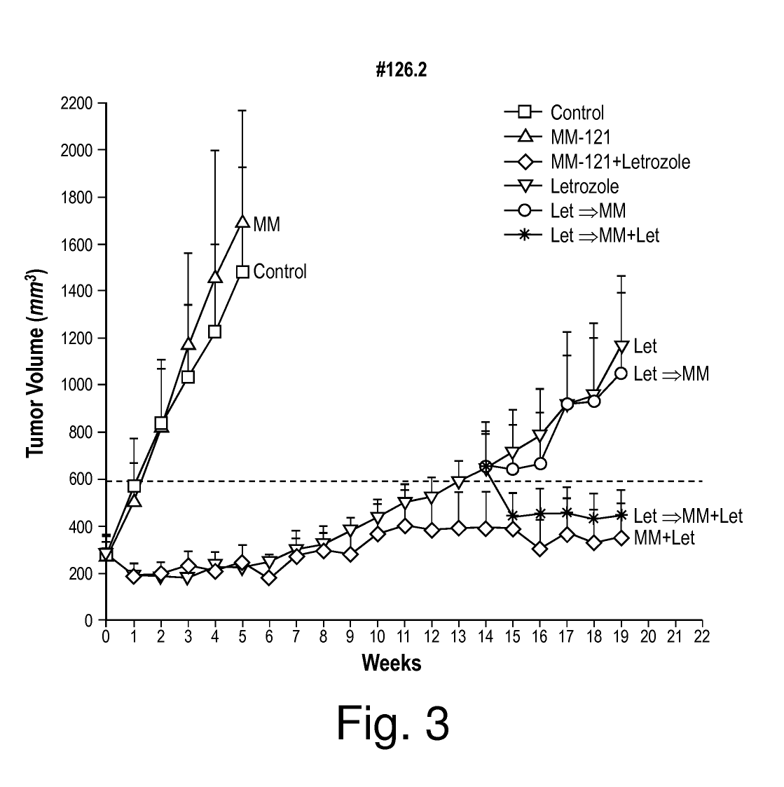

Results are shown in FIG. 3. As indicated therein, the tumors in the MM-121-treated (triangle) and PBS only control (square) mice grew rapidly over the course of 5 weeks, which was the final measurement for these groups. Tumor growth progressed more slowly in the three groups receiving letrozole treatment alone (upside down arrow) and the group receiving combination treatment with MM-121 and letrozole (diamond).

Letrozole resistance in the groups of mice receiving letrozole treatment was defined as the point where tumor volume increased to 600 mm³. This tumor volume was reached in the groups receiving letrozole alone after about 14 weeks of daily letrozole treatment. While the tumors in the mice receiving either letrozole alone or MM-121 alone continued to increase in volume over time, the tumors of the mice receiving the combination of MM-121 and letrozole decreased to well below the 600 mm³ resistance threshold and maintained a reduced volume throughout the rest of the study (19 weeks), thus demonstrating that the combination treatment overcomes acquired letrozole resistance.

In contrast to the mice receiving letrozole alone, the group receiving both letrozole and MM-121 (diamond shape) from the start of treatment did not develop letrozole resistant tumors (i.e. the tumors never reached a volume of 600 mm³), thus demonstrating that treatment with the combination prevents the development of letrozole resistance.

### Example 4

### Co-Administration of MM-121 with an mTOR Inhibitor and an Aromatase Inhibitor

To demonstrate whether the triple combination of MM-121+exemestane+everolimus is more effective than either exemestane alone or the combination of everolimus and exemestane in the treatment of ER+ breast cancer, patients will be dosed with MM-121 alone, exemestane alone, everolimus alone, the combination of everolimus and exemestane, and the combination of MM-121, everolimus, and exemestane. MM-121 will be dosed, e.g., at a 40 mg/kg loading dose on week 1, followed by 20 mg/kg weekly maintenance dose administered over 60 minutes as an intravenous infusion once per week; exemestane will be dosed at 25 mg administered orally once per day; everolimus will be dosed at 10 mg administered orally once per day. Patients will be treated until radiologic or clinical progression of their disease is documented. The results will demonstrate that the triple combination of MM-121+exemestane+everolimus is more effective than exemestane alone or the combination of everolimus and exemestane in the treatment of ER+ breast cancer patients.

### EQUIVALENTS

Those skilled in the art will recognize, or be able to ascertain using no more than routine experimentation, many equivalents of the specific embodiments described herein. Such equivalents are intended to be encompassed by the following claims. Any combinations of the embodiments disclosed in the dependent claims are contemplated to be within the scope of the invention.

### INCORPORATION BY REFERENCE

Each and every, issued patent, patent application and publication referred to herein is hereby incorporated herein by reference in its entirety.

SUMMARY OF SEQUENCE LISTING

MM-121 V_{H} amino acid sequence (SEQ ID NO: 1)
EVQLLESGGGLVQPGGSLRLSCAASGFTFSHYVMAWVRQAPGKGLEWVSSISSSGG
WTLYADSVKGRFTISRDNSKNTLYLQMNSLRAEDTAVYYCTRGLKMATIFDYWGQGTLVT
VSS
MM-121 V_{L} amino acid sequence (SEQ ID NO: 2)
QSALTQPASVSGSPGQSITISCTGTSSDVGSYNVVSWYQQHPGKAPKLIIYEVSQRPSG
VSNRFSGSKSGNTASLTISGLQTEDEADYYCCSYAGSSIFVIFGGGTKVTVL
MM-121 V_{H} CDR1 (SEQ ID NO: 3)
HYVMA
MM-121 V_{H} CDR2 (SEQ ID NO: 4)
SISSSGGWTLYADSVKG
MM-121 V_{H} CDR3 (SEQ ID NO: 5)
GLKMATIFDY
MM-121 V_{L} CDR1 (SEQ ID NO: 6)
TGTSSDVGSYNVVS
MM-121 V_{L} CDR2 (SEQ ID NO: 7)
EVSQRPS
MM-121 V_{L} CDR3 (SEQ ID NO: 8)
CSYAGSSIFVI
Ab # 3 V_{H} amino acid sequence (SEQ ID NO: 9)
EVQLLESGGGLVQPGGSLRLSCAASGFTFSAYNMRWVRQAPGKGLEWVSVIYPSGG
ATRYADSVKGRFTISRDNSKNTLYLQMNSLRAEDTAVYYCARGYYYYGMDVWGQGTLVT
VSS
Ab # 3 V_{L} amino acid sequence (SEQ ID NO: 10)
QSVLTQPPSASGTPGQRVTISCSGSDSNIGRNYIYWYQQFPGTAPKLLIYRNNQRPSGV
PDRISGSKSGTSASLAISGLRSEDEAEYHCGTWDDSLSGPVFGGGTKLTVL
Ab # 3 V_{H} CDR1 (SEQ ID NO: 11)
AYNMR
Ab # 3 V_{H} CDR2 (SEQ ID NO: 12)
VIYPSGGATRYADSVKG
Ab # 3 V_{H} CDR3 (SEQ ID NO: 13)
GYYYYGMDV
Ab # 3 V_{L} CDR1 (SEQ ID NO: 14)
SGSDSNIGRNYIY
Ab # 3 V_{L} CDR2 (SEQ ID NO: 15)
RNNQRPS
Ab # 3 V_{L} CDR3 (SEQ ID NO: 16)
GTWDDSLSGPV
Ab # 14 V_{H} amino acid sequence (SEQ ID NO: 17)
EVQLLESGGGLVQPGGSLRLSCAASGFTFSAYGMGWVRQAPGKGLEWVSYISPSGG
HTKYADSVKGRFTISRDNSKNTLYLQMNSLRAEDTAVYYCAKVLETGLLVDAFDIWGQGT
MVTVSS
Ab # 14 V_{L} amino acid sequence (SEQ ID NO: 18)
QYELTQPPSVSVYPGQTASITCSGDQLGSKFVSWYQQRPGQSPVLVMYKDKRRPSEIP
ERFSGSNSGNTATLTISGTQAIDEADYYCQAWDSSTYVFGTGTKVTVL
Ab # 14 V_{H} CDR1 (SEQ ID NO: 19)
AYGMG
Ab # 14 V_{H} CDR2 (SEQ ID NO: 20)
YISPSGGHTKYADSVKG
Ab # 14 V_{H} CDR3 (SEQ ID NO: 21)
VLETGLLVDAFDI
Ab # 14 V_{L} CDR1 (SEQ ID NO: 22)
SGDQLGSKFVS
Ab # 14 V_{L} CDR2 (SEQ ID NO: 23)
YKDKRRPS
Ab # 14 V_{L} CDR3 (SEQ ID NO: 24)
QAWDSSTYV
Ab # 17 V_{H} amino acid sequence (SEQ ID NO: 25)
EVQLLESGGGLVQPGGSLRLSCAASGFTFSWYGMGWVRQAPGKGLEWVSYISPSGG
ITVYADSVKGRFTISRDNSKNTLYLQMNSLRAEDTAVYYCARLNYYYGLDVWGQGTTVTVS
S
Ab # 17 V_{L} amino acid sequence (SEQ ID NO: 26)
QDIQMTQSPSSLSASVGDRITITCQASQDIGDSLNWYQQKPGKAPRLLIYDASNLETG
VPPRFSGSGSGTDFTFTFRSLQPEDIATYFCQQSANAPFTFGPGTKVDIK
Ab # 17 V_{H} CDR1 (SEQ ID NO: 27)
WYGMG
Ab # 17 V_{H} CDR2 (SEQ ID NO: 28)
YISPSGGITVYADSVKG
Ab # 17 V_{H} CDR3 (SEQ ID NO: 29)
LNYYYGLDV
Ab # 17 V_{L} CDR1 (SEQ ID NO: 30)
QASQDIGDSLN
Ab # 17 V_{L} CDR2 (SEQ ID NO: 31)
DASNLET
Ab # 17 V_{L} CDR3 (SEQ ID NO: 32)
QQSANAPFT
Ab # 19 V_{H} amino acid sequence (SEQ ID NO: 33)
EVQLLESGGGLVQPGGSLRLSCAASGFTFSRYGMWWVRQAPGKGLEWVSYIGSSGG
PTYYVDSVKGRFTISRDNSKNTLYLQMNSLRAEDTAVYYCAGGRGTPYYFDSWGQGTLVTV
SS
Ab # 19 V_{L} amino acid sequence (SEQ ID NO: 34)
QYELTQPASVSGSPGQSITISCTGTSSDIGRWNIVSWYQQHPGKAPKLMIYDVSNRPS
GVSNRF
SGSKSGNTASLTISGLQAEDEADYYCSSYTSSSTWVFGGGTKLTVL
Ab # 19 V_{H} CDR1 (SEQ ID NO: 35)
RYGMW
Ab # 19 V_{H} CDR2 (SEQ ID NO: 36)
YIGSSGGPTYYVDSVKG
Ab # 19 V_{H} CDR3 (SEQ ID NO: 37)
GRGTPYYFDS
Ab # 19 V_{L} CDR1 (SEQ ID NO: 38
TGTSSDIGRWNIVS
Ab # 19 V_{L} CDR2 (SEQ ID NO: 39)
DVSNRPS
Ab # 19 V_{L} CDR3 (SEQ ID NO: 40)
SSYTSSSTWV
ErbB3 (SEQ ID NO: 41)
SEVGNSQAVCPGTLNGLSVTGDAENQYQTLYKLYERCEVVMGNLEIVLTGHNADLSFLQWI
REVTGYVLVAMNEFSTLPLPNLRVVRGTQVYDGKFAIFVMLNYNTNSSHALRQLRLTQLTEI
LSGGVYIEKNDKLCHMDTIDWRDIVRDRDAEIVVKDNGRSCPPCHEVCKGRCWGPGSEDCQ
TLTKTICAPQCNGHCFGPNPNQCCHDECAGGCSGPQDTDCFACRHFNDSGACVPRCPQPLVY
NKLTFQLEPNPHTKYQYGGVCVASCPHNFVVDQTSCVRACPPDKMEVDKNGLKMCEPCGG
LCPKACEGTGSGSRFQTVDSSNIDGFVNCTKILGNLDFLITGLNGDPWHKIPALDPEKLNVFR
TVREITGYLNIQSWPPHMHNFSVFSNLTTIGGRSLYNRGFSLLIMKNLNVTSLGFRSLKEISAG
RIYISANRQLCYHHSLNWTKVLRGPTEERLDIKHNRPRRDCVAEGKVCDPLCSSGGCWGPGP
GQCLSCRNYSRGGVCVTHCNFLNGEPREFAHEAECFSCHPECQPMEGTATCNGSGSDTCAQ
CAHFRDGPHCVSSCPHGVLGAKGPIYKYPDVQNECRPCHENCTQGCKGPELQDCLGQTLVLI
GKTHLTMALTVIAGLVVIFMMLGGTFLYWRGRRIQNKRAMRRYLERGESIEPLDPSEKANK
VLARIFKETELRKLKVLGSGVFGTVHKGVWIPEGESIKIPVCIKVIEDKSGRQSFQAVTDHML
AIGSLDHAHIVRLLGLCPGSSLQLVTQYLPLGSLLDHVRQHRGALGPQLLLNVVGVQIAKGMY
YLEEHGMVHRNLAARNVLLKSPSQVQVADFGVADLLPPDDKQLLYSEAKTPIKWMALESIH
FGKYTHQSDVWSYGVTVWELMTFGAEPYAGLRLAEVPDLLEKGERLAQPQICTIDVYMVM
VKCWMIDENIRPTFKELANEFTRMARDPPRYLVIKRESGPGIAPGPEPHGLTNKKLEEVELEP
ELDLDLDLEAEEDNLATTTLGSALSLPVGTLNRPRGSQSLLSPSSGYMPMNQGNLGESCQES
AVSGSSERCPRPVSLHPMPRGCLASESSEGHVTGSEAELQEKVSMCRSRSRSRSPRPRGDSAY
HSQRHSLLTPVTPLSPPGLEEEDVNGYVMPDTHLKGTPSSREGTLSSVGLSSVLGTEEEDEDE
EYEYMNRRRRHSPPHPPRPSSLEELGYEYMDVGSDLSASLGSTQSCPLHPVPIMPTAGTTPDE
DYEYMNRQRDGGGPGGDYAAMGACPASEQGYEEMRAFQGPGHQAPHVHYARLKTLRSLE
ATDSAFDNPDYWHSRLFPKANAQRT
MM-121 Heavy Chain Amino Acid Sequence (SEQ ID NO: 42)

| | |
|---|---|
| 1 | EVQLLESGGG LVQPGGSLRL SCAASGFTFS HYVMAWVRQA PGKGLEWVSS |
| 51 | ISSSGGWTLY ADSVKGRFTI SRDNSKNTLY LQMNSLRAED TAVYYCTRGL |
| 101 | KMATIFDYWG QGTLVTVSSA STKGPSVFPL APCSRSTSES TAALGCLVKD |
| 151 | YFPEPVTVSW NSGALTSGVH TFPAVLQSSG LYSLSSVVTV PSSNFGTQTY |
| 201 | TCNVDHKPSN TKVDKTVERK CCVECPPCPA PPVAGPSVFL FPPKPKDTLM |
| 251 | ISRTPEVTCV VVDVSHEDPE VQFNWYVDGV EVHNAKTKPR EEQFNSTFRV |
| 301 | VSVLTVVHQD WLNGKEYKCK VSNKGLPAPI EKTISKTKGQ PREPQVYTLP |
| 351 | PSREEMTKNQ VSLTCLVKGF YPSDIAVEWE SNGQPENNYK TTPPMLDSDG |
| 401 | SFFLYSKLTV DKSRWQQGNV FSCSVMHEAL HNHYTQKSLS LSPGK |

MM-121 Light Chain Amino Acid Sequence (SEQ ID NO: 43)

| | |
|---|---|
| 1 | QSALTQPASV SGSPGQSITI SCTGTSSDVG SYNVVSWYQQ HPGKAPKLII |
| 51 | YEVSQRPSGV SNRFSGSKSG NTASLTISGL QTEDEADYYC CSYAGSSIFV |
| 101 | IFGGGTKVTV LGQPKAAPSV TLFPPSSEEL QANKATLVCL VSDFYPGAVT |
| 151 | VAWKADGSPV KVGVETTKPS KQSNNKYAAS SYLSLTPEQW KSHRSYSCRV |
| 201 | THEGSTVEKT VAPAECS |

Bispecific antibody described in US patent publication 20110059076
as SEQ ID NO: 16 (SEQ ID NO: 44)
QVQLQESGGGLVKPGGSLRLSCAASGFTFSSYWMSWVRQAPGKGLEWVANINRDGSASYY
VDSVKGRFTISRDDAKNSLYLQMNSLRAEDTAVYYCARDRGVGYFDLWGRGTLVTVSSAST
GGGGSGGGGSGGGGSQSALTQPASVSGSPGQSITISCTGTSSDVGGYNFVSWYQQHPGKAPK
LMIYDVSDRPSGVSDRFSGSKSGNTASLIISGLQADDEADYYCSSYGSSSTHVIFGGGTKVTVL
GAASDAHKSEVAHRFKDLGEENFKALVLIAFAQYLQQSPFEDHVKLVNEVTEFAKTCVADE
SAENCDKSLHTLFGDKLCTVATLRETYGEMADCCAKQEPERNECFLQHKDDNPNLPRLVRP
EVDVMCTAFHDNEETFLKKYLYEIARRHPYFYAPELLFFAKRYKAAFTECCQAADKAACLLP
KLDELRDEGKASSAKQRLKCASLQKFGERAFKAWAVARLSQRFPKAEFAEVSKLVTDLTKV
HTECCHGDLLECADDRADLAKYICENQDSISSKLKECCEKPLLEKSHCIAEVENDEMPADLPS
LAADFVESKDVCKNYAEAKDVFLGMFLYEYARRHPDYSVVLLLRLAKTYETTLEKCCAAA
DPHECYAKVFDEFKPLVEEPQNLIKQNCELFEQLGEYKFQNALLVRYTKKVPQVSTPTLVEV
SRNLGKVGSKCCKHPEAKRMPCAEDYLSVVLNQLCVLHEKTPVSDRVTKCCTESLVNRRPC
FSALEVDETYVPKEFQAETFTFHADICTLSEKERQIKKQTALVELVKHKPKATKEQLKAVMD
DFAAFVEKCCKADDKETCFAEEGKKLVAASQAALGLAAALQVQLVQSGAEVKKPGESLKIS
CKGSGYSFTSYWIAWVRQMPGKGLEYMGLIYPGDSDTKYSPSFQGQVTISVDKSVSTAYLQ
WSSLKPSDSAVYFCARHDVGYCTDRTCAKWPEWLGVWGQGTLVTVSSGGGGSSGGGSGG
GGSQSVLTQPPSVSAAPGQKVTISCSGSSSNIGNNYVSWYQQLPGTAPKLLIYDHTNRPAGVP
DRFSGSKSGTSASLAISGFRSEDEADYYCASWDYTLSGWVFGGGTKLTVLG

1. (canceled)

2. A method of suppressing growth of an estrogen receptor positive or negative hormone refractory breast cancer cell, the method comprising contacting the cell with an effective amount of an ErbB3 inhibitor.

3. A method of treating or suppressing growth of an estrogen receptor positive or negative hormone refractory breast cancer tumor in a patient, the method comprising administering to the patient an effective amount of an ErbB3 inhibitor.

4. (canceled)

5. A method of treating a breast cancer tumor in a patient, the method comprising
selecting a patient with an estrogen receptor positive or negative hormone refractory breast cancer tumor; and
administering to the patient an effective amount of an ErbB3 inhibitor.

6. The method of claim 2, wherein the ErbB3 inhibitor is an anti-ErbB3 antibody.

7. The method of claim 6, wherein the anti-ErbB3 antibody comprises:
(a) V_{H} and/or V_{L} regions comprising the amino acid sequences set forth in SEQ ID NOs: 1 and 2, respectively;
(b) V_{H} and/or V_{L} regions comprising the amino acid sequences set forth in SEQ ID NOs: 42 and 43, respectively; or
(c) V_{H} CDR1, 2 and 3 sequences as shown in SEQ ID NOs: 3-5, respectively, and, in amino terminal to carboxy terminal order, V_{L} CDR1, 2 and 3 sequences as shown in SEQ ID NOs: 6-8, respectively.

8-9. (canceled)

10. The method of claim 3, further comprising co-administering to the patient an effective amount of at least one additional anti-cancer agent that is not an ErbB3 inhibitor.

11. (canceled)

12. The method of claim 10, wherein the at least one additional anti-cancer agent is selected from the group consisting of platinum-based chemotherapy drugs, taxanes, tyrosine kinase inhibitors, serine/threonine protein kinase inhibitors, anti-EGFR antibodies, anti-ErbB2 antibodies, and combinations thereof.

13-14. (canceled)

15. The method of claim 12, wherein the anti-EGFR antibody comprises cetuximab.

16. The method of claim 10, wherein the at least one additional anti-cancer agent is a VEGF inhibitor or a small molecule inhibitor of EGFR signaling selected from the group consisting of afatinib gefitinib, lapatinib, canertinib, pelitinib, erlotinib, PKI-166, PD158780, and AG 1478.

17-18. (canceled)

19. The method of claim 16, wherein the VEGF inhibitor is bevacizumab.

20. (canceled)

21. The method of claim 3, wherein the hormone refractory breast cancer is estrogen receptor positive (ER+).

22. The method of claim 21 further comprising co-administration of either or both of:
(a) an estrogen receptor antagonist and an aromatase inhibitor; or
(b) an mTOR inhibitor and an aromatase inhibitor.

23. The method of claim 22, wherein:
(a) the estrogen receptor antagonist is selected from raloxifene, tamoxifen, afimoxifene (4-hydroxytamoxifen), arzoxifene, lasofoxone, toremifene and fulvestrant; and the aromatase inhibitor is selected from exemestane, anastrozole, letrozole, aminoglutethimide, testolactone, vorozole, formestane and fadrozole; and
(b) the aromatase inhibitor is letrozole or exemestane.

24-25. (canceled)

26. The method of claim 22, wherein:
(a) the mTOR inhibitor is selected from temsirolimus, everolimus, sirolimus, and ridaforolimus; and the an aromatase inhibitor is selected from exemestane, anastrozole, letrozole, aminoglutethimide, testolactone, vorozole, formestane and fadrozole; and
(b) the aromatase inhibitor is exemestane.

27-28. (canceled)

29. A method of inhibiting heregulin-mediated activation of estrogen receptors in tumor cells, said method comprising 1) selecting a human patient who has been treated for a malignancy with an anti-estrogen therapy and has become resistant to such therapy, which patient has a malignant tumor, which tumor, by analysis of a tumor biopsy taken from the patent after the patient has become resistant, is estrogen receptor positive and overexpresses HER2, and which activation comprises phosphorylation of estrogen receptors, and 2) administering to the patient so selected an antibody that inhibits heregulin binding to ErbB2/ErbB3 heterodimer, wherein the antibody is administered at a dosage that yields a concentration of the antibody in the patient's bloodstream that is a sufficient concentration to inhibit heregulin-induced estrogen receptor phosphorylation in MCF7 cells in vitro by at least 20%, at least 30%, at least 40%, at least 50%, at least 60%, or at least 70%, wherein said administration at said dosage is effective to treat the tumor.

30. The method of claim 29, wherein the malignant tumor is a tumor of the breast, ovary lung, or skin.

31-33. (canceled)

34. The method of claim 29, wherein the antibody is an anti-ErbB2 antibody.

35. The method of claim 29, wherein the antibody is an anti-ErbB3 antibody.

36. The method of claim 35, wherein the antibody comprises:
(a) V_{H} and/or V_{L} regions comprising the amino acid sequences set forth in SEQ ID NOs: 1 and 2, respectively or
(b) V_{H} and/or V_{L} regions comprising the amino acid sequences set forth in SEQ ID NOs: 42 and 43, respectively.

37. (canceled)

38. The method of claim 29, wherein the antibody is an anti-ErbB2/antiErbB3 bispecific antibody.

39-43. (canceled)

44. A composition for inhibition of heregulin-mediated activation of estrogen receptor, said inhibition following selection of a human patient who has been treated for a malignancy with an anti-estrogen therapy and has become resistant to such therapy, which patient has a malignant tumor, which tumor, by analysis of a tumor biopsy taken from the patient after the patient has become resistant, is estrogen receptor positive and overexpresses ErbB2, and which activation comprises phosphorylation of estrogen receptors, said composition comprising:
(a) an anti-ErbB3 antibody that inhibits heregulin binding to ErbB2/ErbB3 heterodimer;
(b) an anti-ErbB2 antibody that binds to ErbB2 and inhibits heregulin binding to ErbB2/ErbB3 heterodimer; or
(c) an anti-ErbB2/antiErbB3 bispecific antibody that inhibits heregulin binding to ErbB2/ErbB3 heterodimer.

45-47. (canceled)

48. A composition for treatment of a hormone refractory estrogen-receptor positive cancer, said composition comprising:
(a) an anti-ErbB3 antibody that inhibits heregulin binding to ErbB2/ErbB3 heterodimer;
(b) an anti-ErbB2 antibody that binds to ErbB2 and inhibits heregulin binding to ErbB2/ErbB3 heterodimer; or
(c) an anti-ErbB2/antiErbB3 bispecific antibody that inhibits heregulin binding to ErbB2/ErbB3 heterodimer.

49-60. (canceled)


SEQUENCE LISTING (44 sequences)

SEQ ID NO: 1 - protein, 119 aa, Homo sapiens [sequence omitted]

SEQ ID NO: 2 - protein, 111 aa, Homo sapiens [sequence omitted]

SEQ ID NO: 3 - protein, 5 aa, Homo sapiens
HYVMA

SEQ ID NO: 4 - protein, 17 aa, Homo sapiens
SISSSGGWTLYADSVKG

SEQ ID NO: 5 - protein, 10 aa, Homo sapiens
GLKMATIFDY

SEQ ID NO: 6 - protein, 14 aa, Homo sapiens
TGTSSDVGSYNVVS

SEQ ID NO: 7 - protein, 7 aa, Homo sapiens
EVSQRPS

SEQ ID NO: 8 - protein, 11 aa, Homo sapiens
CSYAGSSIFVI

SEQ ID NO: 9 - protein, 118 aa, Homo sapiens
EVQLLESGGGLVQPGGSLRLSCAASGFTFSAYNMRWVRQAPGKGLEWVSVIYPSGGATRYADSVKGRFTISRDNSKNTLYLQMNSLRAEDTAVYYCARGYYYYGMDVWGQGTLVTVSS

SEQ ID NO: 10 - protein, 110 aa, Homo sapiens
QSVLTQPPSASGTPGQRVTISCSGSDSNIGRNYIYWYQQFPGTAPKLLIYRNNQRPSGVPDRISGSKSGTSASLAISGLRSEDEAEYHCGTWDDSLSGPVFGGGTKLTVL

SEQ ID NO: 11 - protein, 5 aa, Homo sapiens
AYNMR

SEQ ID NO: 12 - protein, 17 aa, Homo sapiens
VIYPSGGATRYADSVKG

SEQ ID NO: 13 - protein, 9 aa, Homo sapiens
GYYYYGMDV

SEQ ID NO: 14 - protein, 13 aa, Homo sapiens
SGSDSNIGRNYIY

SEQ ID NO: 15 - protein, 7 aa, Homo sapiens
RNNQRPS

SEQ ID NO: 16 - protein, 11 aa, Homo sapiens
GTWDDSLSGPV

SEQ ID NO: 17 - protein, 122 aa, Homo sapiens
EVQLLESGGGLVQPGGSLRLSCAASGFTFSAYGMGWVRQAPGKGLEWVSYISPSGGHTKYADSVKGRFTISRDNSKNTLYLQMNSLRAEDTAVYYCAKVLETGLLVDAFDIWGQGTMVTVSS

SEQ ID NO: 18 - protein, 106 aa, Homo sapiens
QYELTQPPSVSVYPGQTASITCSGDQLGSKFVSWYQQRPGQSPVLVMYKDKRRPSEIPERFSGSNSGNTATLTISGTQAIDEADYYCQAWDSSTYVFGTGTKVTVL

SEQ ID NO: 19 - protein, 5 aa, Homo sapiens
AYGMG

SEQ ID NO: 20 - protein, 17 aa, Homo sapiens
YISPSGGHTKYADSVKG

SEQ ID NO: 21 - protein, 13 aa, Homo sapiens
VLETGLLVDAFDI

SEQ ID NO: 22 - protein, 11 aa, Homo sapiens
SGDQLGSKFVS

SEQ ID NO: 23 - protein, 8 aa, Homo sapiens
YKDKRRPS

SEQ ID NO: 24 - protein, 9 aa, Homo sapiens
QAWDSSTYV

SEQ ID NO: 25 - protein, 118 aa, Homo sapiens
EVQLLESGGGLVQPGGSLRLSCAASGFTFSWYGMGWVRQAPGKGLEWVSYISPSGGITVYADSVKGRFTISRDNSKNTLYLQMNSLRAEDTAVYYCARLNYYYGLDVWGQGTTVTVSS

SEQ ID NO: 26 - protein, 108 aa, Homo sapiens
QDIQMTQSPSSLSASVGDRITITCQASQDIGDSLNWYQQKPGKAPRLLIYDASNLETGVPPRFSGSGSGTDFTFTFRSLQPEDIATYFCQQSANAPFTFGPGTKVDIK

SEQ ID NO: 27 - protein, 5 aa, Homo sapiens
WYGMG

SEQ ID NO: 28 - protein, 17 aa, Homo sapiens
YISPSGGITVYADSVKG

SEQ ID NO: 29 - protein, 9 aa, Homo sapiens
LNYYYGLDV

SEQ ID NO: 30 - protein, 11 aa, Homo sapiens
QASQDIGDSLN

SEQ ID NO: 31 - protein, 7 aa, Homo sapiens
DASNLET

SEQ ID NO: 32 - protein, 9 aa, Homo sapiens
QQSANAPFT

SEQ ID NO: 33 - protein, 119 aa, Homo sapiens
EVQLLESGGGLVQPGGSLRLSCAASGFTFSRYGMWWVRQAPGKGLEWVSYIGSSGGPTYYVDSVKGRFTISRDNSKNTLYLQMNSLRAEDTAVYYCAGGRGTPYYFDSWGQGTLVTVSS

SEQ ID NO: 34 - protein, 110 aa, Homo sapiens
QYELTQPASVSGSPGQSITISCTGTSSDIGRWNIVSWYQQHPGKAPKLMIYDVSNRPSGVSNRFSGSKSGNTASLTISGLQAEDEADYYCSSYTSSSTWVFGGGTKLTVL

SEQ ID NO: 35 - protein, 5 aa, Homo sapiens
RYGMW

SEQ ID NO: 36 - protein, 17 aa, Homo sapiens
YIGSSGGPTYYVDSVKG

SEQ ID NO: 37 - protein, 10 aa, Homo sapiens
GRGTPYYFDS

SEQ ID NO: 38 - protein, 14 aa, Homo sapiens
TGTSSDIGRWNIVS

SEQ ID NO: 39 - protein, 7 aa, Homo sapiens
DVSNRPS

SEQ ID NO: 40 - protein, 10 aa, Homo sapiens
SSYTSSSTWV

SEQ ID NO: 41 - protein, 1323 aa, Homo sapiens
SEVGNSQAVCPGTLNGLSVTGDAENQYQTLYKLYERCEVVMGNLEIVLTGHNADLSFLQWIREVTGYVLVAMNEFSTLPLPNLRVVRGTQVYDGKFAIFVMLNYNTNSSHALRQLRLTQLTEILSGGVYIEKNDKLCHMDTIDWRDIVRDRDAEIVVKDNGRSCPPCHEVCKGRCWGPGSEDCQTLTKTICAPQCNGHCFGPNPNQCCHDECAGGCSGPQDTDCFACRHFNDSGACVPRCPQPLVYNKLTFQLEPNPHTKYQYGGVCVASCPHNFVVDQTSCVRACPPDKMEVDKNGLKMCEPCGGLCPKACEGTGSGSRFQTVDSSNIDGFVNCTKILGNLDFLITGLNGDPWHKIPALDPEKLNVFRTVREITGYLNIQSWPPHMHNFSVFSNLTTIGGRSLYNRGFSLLIMKNLNVTSLGFRSLKEISAGRIYISANRQLCYHHSLNWTKVLRGPTEERLDIKHNRPRRDCVAEGKVCDPLCSSGGCWGPGPGQCLSCRNYSRGGVCVTHCNFLNGEPREFAHEAECFSCHPECQPMEGTATCNGSGSDTCAQCAHFRDGPHCVSSCPHGVLGAKGPIYKYPDVQNECRPCHENCTQGCKGPELQDCLGQTLVLIGKTHLTMALTVIAGLVVIFMMLGGTFLYWRGRRIQNKRAMRRYLERGESIEPLDPSEKANKVLARIFKETELRKLKVLGSGVFGTVHKGVWIPEGESIKIPVCIKVIEDKSGRQSFQAVTDHMLAIGSLDHAHIVRLLGLCPGSSLQLVTQYLPLGSLLDHVRQHRGALGPQLLLNWGVQIAKGMYYLEEHGMVHRNLAARNVLLKSPSQVQVADFGVADLLPPDDKQLLYSEAKTPIKWMALESIHFGKYTHQSDVWSYGVTVWELMTFGAEPYAGLRLAEVPDLLEKGERLAQPQICTIDVYMVMVKCWMIDENIRPTFKELANEFTRMARDPPRYLVIKRESGPGIAPGPEPHGLTNKKLEEVELEPELDLDLDLEAEEDNLATTTLGSALSLPVGTLNRPRGSQSLLSPSSGYMPMNQGNLGESCQESAVSGSSERCPRPVSLHPMPRGCLASESSEGHVTGSEAELQEKVSMCRSRSRSRSPRPRGDSAYHSQRHSLLTPVTPLSPPGLEEEDVNGYVMPDTHLKGTPSSREGTLSSVGLSSVLGTEEEDEDEEYEYMNRRRRHSPPHPPRPSSLEELGYEYMDVGSDLSASLGSTQSCPLHPVPIMPTAGTTPDEDYEYMNRQRDGGGPGGDYAAMGACPASEQGYEEMRAFQGPGHQAPHVHYARLKTLRSLEATDSAFDNPDYWHSRLFPKANAQRT

SEQ ID NO: 42 - protein, 445 aa, Homo sapiens
EVQLLESGGGLVQPGGSLRLSCAASGFTFSHYVMAWVRQAPGKGLEWVSSISSSGGWTLYADSVKGRFTISRDNSKNTLYLQMNSLRAEDTAVYYCTRGLKMATIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 43 - protein, 217 aa, Homo sapiens
QSALTQPASVSGSPGQSITISCTGTSSDVGSYNVVSWYQQHPGKAPKLIIYEVSQRPSGVSNRFSGSKSGNTASLTISGLQTEDEADYYCCSYAGSSIFVIFGGGTKVTVLGQPKAAPSVTLFPPSSEELQANKATLVCLVSDFYPGAVTVAWKADGSPVKVGVETTKPSKQSNNKYAASSYLSLTPEQWKSHRSYSCRVTHEGSTVEKTVAPAECS

SEQ ID NO: 44 - protein, 1095 aa, Artificial Sequence
  source: /note=“Description of Artificial Sequence Synthetic polypeptide”
QVQLQESGGGLVKPGGSLRLSCAASGFTFSSYWMSWVRQAPGKGLEWVANINRDGSASYYVDSVKGRFTISRDDAKNSLYLQMNSLRAEDTAVYYCARDRGVGYFDLWGRGTLVTVSSASTGGGGSGGGGSGGGGSQSALTQPASVSGSPGQSITISCTGTSSDVGGYNFVSWYQQHPGKAPKLMIYDVSDRPSGVSDRFSGSKSGNTASLIISGLQADDEADYYCSSYGSSSTHVIFGGGTKVTVLGAASDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFQAETFTFHADICTLSEKERQIKKQTALVELVKHKPKATKEQLKAVMDDFAAFVEKCCKADDKETCFAEEGKKLVAASQAALGLAAALQVQLVQSGAEVKKPGESLKISCKGSGYSFTSYWIAWVRQMPGKGLEYMGLIYPGDSDTKYSPSFQGQVTISVDKSVSTAYLQWSSLKPSDSAVYFCARHDVGYCTDRTCAKWPEWLGVWGQGTLVTVSSGGGGSSGGGSGGGGSQSVLTQPPSVSAAPGQKVTISCSGSSSNIGNNYVSWYQQLPGTAPKLLIYDHTNRPAGVPDRFSGSKSGTSASLAISGFRSEDEADYYCASWDYTLSGWVFGGGTKLTVLG